SEQUENCE LISTING (56 sequences)

SEQ ID NO: 1 - protein, 74 aa, Bacillus cereus
  cerein 7B with signal pept
MENLQMLTEEELMEIEGGGWWNSWGKCVAGTIGGAGTGGLGGAAAGSAVPVIGTGIGGAIGGVSGGLTGAATFC

SEQ ID NO: 2 - protein, 56 aa, Bacillus cereus
  cerein 7B
GWWNSWGKCVAGTIGGAGTGGLGGAAAGSAVPVIGTGIGGAIGGVSGGLTGAATFC

SEQ ID NO: 3 - protein, 56 aa, Artificial sequence
  bacteriocin consensus
  misc_feature (4)..(5): Xaa represents any amino acid
  misc_feature (11)..(11): Xaa represents any amino acid
  misc_feature (13)..(13): Xaa represents any amino acid
  misc_feature (16)..(18): Xaa represents any amino acid
  misc_feature (21)..(21): Xaa represents any amino acid
  misc_feature (23)..(23): Xaa represents any amino acid
  misc_feature (25)..(27): Xaa represents any amino acid
  misc_feature (30)..(30): Xaa represents any amino acid
  misc_feature (37)..(37): Xaa represents any amino acid
  misc_feature (40)..(41): Xaa represents any amino acid
  misc_feature (44)..(44): Xaa represents any amino acid
  misc_feature (46)..(46): Xaa represents any amino acid
  misc_feature (48)..(48): Xaa represents any amino acid
  misc_feature (51)..(54): Xaa represents any amino acid
GWWXXWGKCVXGXIGXXXTGXLXGXXXGSXVPVIGTXIGXXIGXVXGXLTXXXXFC

SEQ ID NO: 4 - protein, 56 aa, Artificial sequence
  mutant 1 (A2)
AWWNSWGKCVAGTIGGAGTGGLGGAAAGSAVPVIGTGIGGAIGGVSGGLTGAATFC

SEQ ID NO: 5 - protein, 56 aa, Artificial sequence
  mutant 2 (A3)
GAWNSWGKCVAGTIGGAGTGGLGGAAAGSAVPVIGTGIGGAIGGVSGGLTGAATFC

SEQ ID NO: 6 - protein, 56 aa, Artificial sequence
  mutant 3 (A4)
GWANSWGKCVAGTIGGAGTGGLGGAAAGSAVPVIGTGIGGAIGGVSGGLTGAATFC

SEQ ID NO: 7 - protein, 56 aa, Artificial sequence
  mutant 4 (A5)
GWWASWGKCVAGTIGGAGTGGLGGAAAGSAVPVIGTGIGGAIGGVSGGLTGAATFC

SEQ ID NO: 8 - protein, 56 aa, Artificial sequence
  mutant 5 (A6)
GWWNAWGKCVAGTIGGAGTGGLGGAAAGSAVPVIGTGIGGAIGGVSGGLTGAATFC

SEQ ID NO: 9 - protein, 56 aa, Artificial sequence
  mutant 6 (A7)
GWWNSAGKCVAGTIGGAGTGGLGGAAAGSAVPVIGTGIGGAIGGVSGGLTGAATFC

SEQ ID NO: 10 - protein, 56 aa, Artificial sequence
  mutant 7 (A8)
GWWNSWAKCVAGTIGGAGTGGLGGAAAGSAVPVIGTGIGGAIGGVSGGLTGAATFC

SEQ ID NO: 11 - protein, 56 aa, Artificial sequence
  mutant 8 (A9)
GWWNSWGACVAGTIGGAGTGGLGGAAAGSAVPVIGTGIGGAIGGVSGGLTGAATFC

SEQ ID NO: 12 - protein, 56 aa, Artificial sequence
  mutant 9 (A10)
GWWNSWGKAVAGTIGGAGTGGLGGAAAGSAVPVIGTGIGGAIGGVSGGLTGAATFC

SEQ ID NO: 13 - protein, 56 aa, Artificial sequence
  mutant 10 (A11)
GWWNSWGKCAAGTIGGAGTGGLGGAAAGSAVPVIGTGIGGAIGGVSGGLTGAATFC

SEQ ID NO: 14 - protein, 56 aa, Artificial sequence
  mutant 11 (A12)
GWWNSWGKCVAATIGGAGTGGLGGAAAGSAVPVIGTGIGGAIGGVSGGLTGAATFC

SEQ ID NO: 15 - protein, 56 aa, Artificial sequence
  mutant 12 (B1)
GWWNSWGKCVAGAIGGAGTGGLGGAAAGSAVPVIGTGIGGAIGGVSGGLTGAATFC

SEQ ID NO: 16 - protein, 56 aa, Artificial sequence
  mutant 13 (B2)
GWWNSWGKCVAGTAGGAGTGGLGGAAAGSAVPVIGTGIGGAIGGVSGGLTGAATFC

SEQ ID NO: 17 - protein, 56 aa, Artificial sequence
  mutant 14 (B3)
GWWNSWGKCVAGTIAGAGTGGLGGAAAGSAVPVIGTGIGGAIGGVSGGLTGAATFC

SEQ ID NO: 18 - protein, 56 aa, Artificial sequence
  mutant 15 (B4)
GWWNSWGKCVAGTIGAAGTGGLGGAAAGSAVPVIGTGIGGAIGGVSGGLTGAATFC

SEQ ID NO: 19 - protein, 56 aa, Artificial sequence
  mutant 16 (B5)
GWWNSWGKCVAGTIGGAATGGLGGAAAGSAVPVIGTGIGGAIGGVSGGLTGAATFC

SEQ ID NO: 20 - protein, 56 aa, Artificial sequence
  mutant 17 (B6)
GWWNSWGKCVAGTIGGAGAGGLGGAAAGSAVPVIGTGIGGAIGGVSGGLTGAATFC

SEQ ID NO: 21 - protein, 56 aa, Artificial sequence
  mutant 18 (B7)
GWWNSWGKCVAGTIGGAGTAGLGGAAAGSAVPVIGTGIGGAIGGVSGGLTGAATFC

SEQ ID NO: 22 - protein, 56 aa, Artificial sequence
  mutant 19 (B8)
GWWNSWGKCVAGTIGGAGTGALGGAAAGSAVPVIGTGIGGAIGGVSGGLTGAATFC

SEQ ID NO: 23 - protein, 56 aa, Artificial sequence
  mutant 20 (B9)
GWWNSWGKCVAGTIGGAGTGGAGGAAAGSAVPVIGTGIGGAIGGVSGGLTGAATFC

SEQ ID NO: 24 - protein, 56 aa, Artificial sequence
  mutant 21 (B10)
GWWNSWGKCVAGTIGGAGTGGLAGAAAGSAVPVIGTGIGGAIGGVSGGLTGAATFC

SEQ ID NO: 25 - protein, 56 aa, Artificial sequence
  mutant 22 (B11)
GWWNSWGKCVAGTIGGAGTGGLGAAAAGSAVPVIGTGIGGAIGGVSGGLTGAATFC

SEQ ID NO: 26 - protein, 56 aa, Artificial sequence
  mutant 23 (B12)
GWWNSWGKCVAGTIGGAGTGGLGGAAAASAVPVIGTGIGGAIGGVSGGLTGAATFC

SEQ ID NO: 27 - protein, 56 aa, Artificial sequence
  mutant 24 (C1)
GWWNSWGKCVAGTIGGAGTGGLGGAAAGAAVPVIGTGIGGAIGGVSGGLTGAATFC

SEQ ID NO: 28 - protein, 56 aa, Artificial sequence
  mutant 25 (C2)
GWWNSWGKCVAGTIGGAGTGGLGGAAAGSAAPVIGTGIGGAIGGVSGGLTGAATFC

SEQ ID NO: 29 - protein, 56 aa, Artificial sequence
  mutant 26 (C3)
GWWNSWGKCVAGTIGGAGTGGLGGAAAGSAVAVIGTGIGGAIGGVSGGLTGAATFC

SEQ ID NO: 30 - protein, 56 aa, Artificial sequence
  mutant 27 (C4)
GWWNSWGKCVAGTIGGAGTGGLGGAAAGSAVPAIGTGIGGAIGGVSGGLTGAATFC

SEQ ID NO: 31 - protein, 56 aa, Artificial sequence
  mutant 28 (C5)
GWWNSWGKCVAGTIGGAGTGGLGGAAAGSAVPVAGTGIGGAIGGVSGGLTGAATFC

SEQ ID NO: 32 - protein, 56 aa, Artificial sequence
  mutant 29 (C6)
GWWNSWGKCVAGTIGGAGTGGLGGAAAGSAVPVIATGIGGAIGGVSGGLTGAATFC

SEQ ID NO: 33 - protein, 56 aa, Artificial sequence
  mutant 30 (C7)
GWWNSWGKCVAGTIGGAGTGGLGGAAAGSAVPVIGAGIGGAIGGVSGGLTGAATFC

SEQ ID NO: 34 - protein, 56 aa, Artificial sequence
  mutant 31 (C8)
GWWNSWGKCVAGTIGGAGTGGLGGAAAGSAVPVIGTAIGGAIGGVSGGLTGAATFC

SEQ ID NO: 35 - protein, 56 aa, Artificial sequence
  mutant 32 (C9)
GWWNSWGKCVAGTIGGAGTGGLGGAAAGSAVPVIGTGAGGAIGGVSGGLTGAATFC

SEQ ID NO: 36 - protein, 56 aa, Artificial sequence
  mutant 33 (C10)
GWWNSWGKCVAGTIGGAGTGGLGGAAAGSAVPVIGTGIAGAIGGVSGGLTGAATFC

SEQ ID NO: 37 - protein, 56 aa, Artificial sequence
  mutant 34 (C11)
GWWNSWGKCVAGTIGGAGTGGLGGAAAGSAVPVIGTGIGAAIGGVSGGLTGAATFC

SEQ ID NO: 38 - protein, 56 aa, Artificial sequence
  mutant 35 (C12)
GWWNSWGKCVAGTIGGAGTGGLGGAAAGSAVPVIGTGIGGAAGGVSGGLTGAATFC

SEQ ID NO: 39 - protein, 56 aa, Artificial sequence
  mutant 36 (D1)
GWWNSWGKCVAGTIGGAGTGGLGGAAAGSAVPVIGTGIGGAIAGVSGGLTGAATFC

SEQ ID NO: 40 - protein, 56 aa, Artificial sequence
  mutant 37 (D2)
GWWNSWGKCVAGTIGGAGTGGLGGAAAGSAVPVIGTGIGGAIGAVSGGLTGAATFC

SEQ ID NO: 41 - protein, 56 aa, Artificial sequence
  mutant 38 (D3)
GWWNSWGKCVAGTIGGAGTGGLGGAAAGSAVPVIGTGIGGAIGGASGGLTGAATFC

SEQ ID NO: 42 - protein, 56 aa, Artificial sequence
  mutant 39 (D4)
GWWNSWGKCVAGTIGGAGTGGLGGAAAGSAVPVIGTGIGGAIGGVAGGLTGAATFC

SEQ ID NO: 43 - protein, 56 aa, Artificial sequence
  mutant 40 (D5)
GWWNSWGKCVAGTIGGAGTGGLGGAAAGSAVPVIGTGIGGAIGGVSAGLTGAATFC

SEQ ID NO: 44 - protein, 56 aa, Artificial sequence
  mutant 41 (D6)
GWWNSWGKCVAGTIGGAGTGGLGGAAAGSAVPVIGTGIGGAIGGVSGALTGAATFC

SEQ ID NO: 45 - protein, 56 aa, Artificial sequence
  mutant 42 (D7)
GWWNSWGKCVAGTIGGAGTGGLGGAAAGSAVPVIGTGIGGAIGGVSGGATGAATFC

SEQ ID NO: 46 - protein, 56 aa, Artificial sequence
  mutant 43 (D8)
GWWNSWGKCVAGTIGGAGTGGLGGAAAGSAVPVIGTGIGGAIGGVSGGLAGAATFC

SEQ ID NO: 47 - protein, 56 aa, Artificial sequence
  mutant 44 (D9)
GWWNSWGKCVAGTIGGAGTGGLGGAAAGSAVPVIGTGIGGAIGGVSGGLTAAATFC

SEQ ID NO: 48 - protein, 56 aa, Artificial sequence
  mutant 45 (D10)
GWWNSWGKCVAGTIGGAGTGGLGGAAAGSAVPVIGTGIGGAIGGVSGGLTGAAAFC

SEQ ID NO: 49 - protein, 56 aa, Artificial sequence
  mutant 46 (D11)
GWWNSWGKCVAGTIGGAGTGGLGGAAAGSAVPVIGTGIGGAIGGVSGGLTGAATAC

SEQ ID NO: 50 - protein, 56 aa, Artificial sequence
  mutant 47 (D12)
GWWNSWGKCVAGTIGGAGTGGLGGAAAGSAVPVIGTGIGGAIGGVSGGLTGAATFA

SEQ ID NO: 51 - protein, 57 aa, Artificial sequence
  mutant 48 (E2)
GWWNSWGKCVAGTIGGAGTGGLGGAAAGSAVPVIGTGIGGAIGGVSGGLTGAATFCW

SEQ ID NO: 52 - protein, 57 aa, Bacillus cereus
  class II bacteriocin
GGWWNSWGKCAAGTLGGTGTGVLGGAAVGSSVPVIGTGIGAAVGGVSGALTGAASAC

SEQ ID NO: 53 - protein, 57 aa, Bacillus cereus
  class II bacteriocin
GGWWSSWGKCAAGTLGGTGTGVLGGAAVGSSVPVIGTGIGAAVGGVSGALTGAASAC

SEQ ID NO: 54 - protein, 58 aa, Bacillus thuringiensis
  class II bacteriocin
GGGWWSSWGKCAAGTLGGTGTGALGGAAVGSSVPVIGTGIGAAVGGVSGALTGAAASC

SEQ ID NO: 55 - DNA, 723 bp, Artificial sequence
  cerein 7B
gcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcggatctcgatcccgcgaaattaatacgactcactataggggaattgtgagcggataacaattcccctctagaaataattttgtttaactttaagaaggagatatacatatgggctggtggaacagctggggtaaatgtgttgctggaactatcggtggagctggaactggtggtttaggtggagctgctgcaggttcagctgttccggttattggtactggtattggtggcgctattggtggagttagcggtggccttacaggtgcagctactttttgctaggatccatccggctgctaacaaagcccgaaaggaagctgagttggctgctgccaccgctgagcaataactagcataaccccttggggcctctaaacgggtcttgaggggttttttgctgaaaggaggaactatatccggattcaggagagcgttcaccgacaaacaacagataaaacgaaaggcccagtctttcgactgagcctttcgttttatttgatgcctggcagttccctactctcgcatcacgtg

SEQ ID NO: 56 - protein, 6 aa, Artificial sequence
  motif
GWGDVL

(19) United States
(12) Patent Application Publication
(10) Pub. No.: US 2024/0197820 A1
(43) Pub. Date: Jun. 20, 2024

(54) Bacteriocin for new application

(71) Applicant: Syngulon S.A., Seraing (BE)

(72) Inventors: Philippe Gabant, Ottignies Louvain-La-Neuve (BE); Félix Jaumaux, Seraing (BE)

(21) Appl. No.: 18/566,900

(22) PCT Filed: May 30, 2022
(86) PCT No.: PCT/EP2022/064576

§ 371 Date: Dec. 4, 2023

(30) Foreign Application Priority Data
Jun. 2, 2021 (EP) 21177483.1

(51) Int. Cl. A61K 38/16; A01N 37/46; A01P 1/00; A61K 8/64; A61P 31/04; A61Q 17/00; C07K 14/32

(52) U.S. Cl. CPC A61K 38/164; A01N 37/46; A01P 1/00; A61K 8/64; A61P 31/04; A61Q 17/005; C07K 14/32

(57) ABSTRACT

Described herein are bacteriocin peptides, peptidomimetics, compositions, pharmaceutical compositions, and cosmetic compositions comprising same. Aspects and embodiments described herein may be used in medical treatment, disinfection of surfaces and cosmetics.

### FIELD

Aspects herein generally pertain to the field of antimicrobial compounds, more particularly to bacteriocin peptides and/or peptidomimetics and compositions comprising same. Also encompassed are uses of bacteriocin peptides, peptidomimetics, and/or compositions comprising bacteriocin peptides and/or peptidomimetics in medical treatment, disinfection of surfaces and cosmetics.

### BACKGROUND

Populations of microbial organisms, such as for example the microbiota associated with the gut and skin of humans, and roots of plants are involved in maintaining the health and metabolic functions of multicellular organisms. Accordingly, tuning populations of microbial organisms, for example to reduce or eliminate or neutralize undesired microbial organisms, can be useful for maintaining the health of tissues that comprise microbial organisms.

Extensive and widespread use of antimicrobial compounds to reduce or eliminate or neutralize undesired microbial organisms has led to the emergence of resistant strains of microbial organisms. As a result, these resistant microbial organisms are no longer susceptible to the currently available antimicrobial compounds or disinfection methods. Accordingly, there is still a need for improved antimicrobial compounds and disinfection methods compared to the ones currently available in the art.

Staphylococcus aureus is one of the leading causes of community-acquired infections, as well as healthcare-acquired infections. Strains of this bacterium are often associated with infections of multiple tissues and organs, such as the skin, lungs, CNS, heart, bones, joints, and blood. Said infections can be started by bacteria present on surfaces that come into contact with the skin and/or bacteria normally present on the body if host defences are compromised. The recent emergence and spreading of methicillin-resistant S. aureus (MRSA) and vancomycin-resistant S. aureus (VRSA) has increased the risk of the associated infections and decreased the effectiveness of available treatments (Shorr, 2007; Chan et al., 2015; McGuiness et al., 2017). Accordingly, there is still a need for improved antimicrobial compounds and methods against S. aureus, particularly against MRSA and/or VRSA.

Bacteriocins are proteinaceous (peptidic) toxins produced by microbial organisms, typically to inhibit the growth of microbial organisms other than the producing cell. Bacteriocins are able to overcome at least some of the drawbacks associated with commonly used antimicrobials, as they are often still active against microbial organisms that are resistant to commonly used antimicrobial compounds such as antibiotics. Currently, their application is mainly focused on broad-spectrum bacteriocins alone or in combination with antibiotics to increase their effectiveness (Ovchinnikov et al., 2020). Approaches utilizing broad-spectrum bacteriocins and/or other antimicrobials are detrimental to beneficial bacteria present in the subject's microbiome when applied to treatment or cosmetic methods, which can be detrimental to the host who is losing their beneficial effects. Accordingly, there is still a need for improved antimicrobial compounds and methods that do not share the aforementioned drawbacks.

Oscáriz and co-workers (2005) previously identified the bacteriocin cerein 7B, produced by Bacillus cereus Bc7, and demonstrated its activity against Lactococcus lactis LMG IL 1403.

### SUMMARY

An aspect of the invention relates to a bacteriocin peptide or peptidomimetic, wherein said peptide or peptidomimetic comprises, essentially consists of, or consists of, preferably comprises, a peptide or peptidomimetic represented the amino acid sequence of SEQ ID NO: 2, or by an amino acid sequence having at least 60%, 70%, 80%, 85%, 90%, 95%, or 99% identity or similarity with SED ID NO: 2, for use in the treatment, prevention and/or delaying of an infection on a subject, preferably a human subject, wherein the infection is:
a. a skin infection and/or
b. caused by Staphylococcus aureus.

In some embodiments, the skin infection is caused by a bacterium, preferably a Gram-positive bacterium. In some embodiments, the skin infection is caused by Staphylococcus aureus. In some embodiments, the Staphylococcus aureus is methicillin-resistant.

In some embodiments, the bacteriocin peptide or peptidomimetic for use according to the invention is such that said use does not inhibit at least one of the microbial cells normally present on the subject's skin, preferably Staphylococcus epidermidis.

Another aspect of the invention relates to a composition comprising a bacteriocin peptide or peptidomimetic according to the invention. In some embodiments, the composition is a pharmaceutical composition optionally further comprising one or more antimicrobial compounds and/or pharmaceutically acceptable ingredients. In some embodiments, the composition is a cosmetic composition optionally further comprising one or more antimicrobial compounds and/or cosmetically acceptable ingredients. In some embodiments, the composition is suitable for disinfecting a surface contaminated with Staphylococcus aureus, optionally further comprising one or more antimicrobial compounds and/or a solvent.

Another aspect of the invention relates to a pharmaceutical composition according to the invention for use according to the invention. In some embodiments, a bacteriocin peptide, peptidomimetic, or pharmaceutical composition for use according to the invention is such that said peptide, peptidomimetic, or composition is administered topically or transdermally, preferably on a wound, lesion, or abscess.

Another aspect of the invention relates to a cosmetic method of providing an improvement of skin hygiene comprising applying to the skin a bacteriocin peptide, peptidomimetic, or cosmetic composition according to the invention.

Another aspect of the invention relates to an ex-vivo method of disinfecting a surface comprising contacting said surface with a bacteriocin peptide, peptidomimetic, or composition according to the invention.

Another aspect of the invention relates to a bacteriocin peptide or peptidomimetic which comprises, essentially consists of, or consists of, preferably comprises, a peptide or peptidomimetic represented by the amino acid sequence of any one of SEQ ID NOs: 7, 8, 15, 18, 19, 22, 24, 34, 37, 40, 42, 44, 47, 48, or 51.

### DESCRIPTION

The present inventors have surprisingly found that bacteriocins described herein are able to selectively inhibit microbial cells associated with infections, particularly with skin infections, while not inhibiting other microbial cells normally present in a subject's microbiome. Particularly, as elaborated in the experimental part, the present inventors have surprisingly found that said bacteriocins are able to inhibit Staphylococcus aureus, while not inhibiting Staphylococcus epidermidis. Accordingly, the aspects and embodiments described herein solve at least some of the problems and needs discussed herein.

### Bacteriocin

Bacteriocins are antimicrobial compounds. A “bacteriocin” as used herein has its customary and ordinary meaning as understood by one of skill in the art in view of this disclosure. It refers to proteinaceous (peptidic) toxins produced by bacteria. The biological activity of bacteriocins is the inhibition of microbial cells other than the host cell by which the peptide is made. Said biological activity may be referred to as antimicrobial activity. A bacteriocin may inhibit at least one cell and/or strain other than the host cell and/or strain in which the peptide is made, including cells and/or strains clonally related to the host cell and other microbial cells. Detailed descriptions of bacteriocins, including methods and compositions for using bacteriocins to control the growth of microbial cells can be found, for example, in U.S. Pat. No. 9,333,227, which is hereby incorporated by reference in its entirety.

Cerein 7B is a bacteriocin which is naturally produced by Bacillus cereus, particularly by Bacillus cereus Bc7 (Oscáriz et al., 2005, incorporated herein by reference in its entirety). Cerein 7B is typically naturally synthesized as an inactive peptide that comprises a leader peptide sequence which is cleaved concomitantly with its transport outside the producing cell, resulting in a mature peptide that displays antimicrobial activity. A wild-type cerein 7B open reading frame (ORF) encodes a sequence of 74 amino acids in length (SEQ ID NO: 1, Uniprot Accession No: Q2MDB2), comprising an amino acid leader peptide sequence which has a length of 18 amino acids. A mature cerein 7B peptide may have a length of 56 amino acids and may be represented by the amino acid sequence:

(SEQ ID NO: 2)
GWWNSWGKCVAGTIGGAGTGGLGGAAAGSAVPVIGTGIGGAIGGVSGGL
TGAATFC

Within the context of the invention, a bacteriocin may be a peptide or a peptidomimetic. A peptide as described herein also encompasses polypeptides, as well as variants (mutants) of peptides and polypeptides as described later herein. A definition of “peptidomimetic” is provided later herein. A “peptidomimetic” as described herein also encompasses variants (mutants) of peptidomimetics. as described later herein. A bacteriocin peptide or peptidomimetic as described herein may be comprised in a composition, such as a pharmaceutical composition, optionally further comprising one or more antimicrobial compounds and/or pharmaceutically acceptable ingredients. A description of compositions, such as pharmaceutical compositions, according to the invention is given in the section titled “compositions”.

The present inventors have surprisingly found that bacteriocins described herein exhibit advantageous antimicrobial properties, as they inhibit microbial cells associated with infections, particularly with skin infections, while not inhibiting other microbial cells normally present in a subject's microbiome. In particular, they are able to inhibit Staphylococcus aureus, while not inhibiting Staphylococcus epidermidis.

A bacteriocin peptide or peptidomimetic as described herein may, therefore, advantageously be used for treatment, prevention and/or delaying of an infection, such as but not limited to a skin infection. Preferably, said infection is caused by a bacterium, more preferably by Staphylococcus aureus. Preferably, said use does not inhibit at least one of the microbial cells, preferably a bacterium, normally present in a subject's microbiome such as, for example, the skin microbiome, more preferably it does not inhibit Staphylococcus epidermidis.

In a first aspect, the invention provides a bacteriocin peptide, peptidomimetic, or a pharmaceutical composition comprising a bacteriocin peptide or peptidomimetic, optionally further comprising one or more antimicrobial compounds and/or pharmaceutically acceptable ingredients, wherein said peptide or peptidomimetic comprises, essentially consists of, or consists of, preferably comprises, a peptide or peptidomimetic represented by the amino acid sequence of SEQ ID NO: 2, or by an amino acid sequence having at least 60%, 70%, 80%, 85%, 90%, 95%, or 99% identity or similarity with SED ID NO: 2, for use in the treatment, prevention and/or delaying of an infection on a subject wherein the infection is:
a. a skin infection and/or
b. caused by Staphylococcus aureus.

In an aspect, the invention provides a method for treating, preventing and/or delaying an infection comprising administering a bacteriocin peptide, peptidomimetic, or a pharmaceutical composition comprising a bacteriocin peptide or peptidomimetic, optionally further comprising one or more antimicrobial compounds and/or pharmaceutically acceptable ingredients, to a subject such as a subject in need thereof, wherein said peptide or peptidomimetic comprises, essentially consists of, or consists of, preferably comprises, a peptide or peptidomimetic represented by the amino acid sequence of SEQ ID NO: 2, or by an amino acid sequence having at least 60%, 70%, 80%, 85%, 90%, 95%, or 99% identity or similarity with SED ID NO: 2, wherein the infection is:
a. a skin infection and/or
b. caused by Staphylococcus aureus.

In an aspect, the invention provides the use of a bacteriocin peptide, peptidomimetic, or a pharmaceutical composition comprising a bacteriocin peptide or peptidomimetic, optionally further comprising one or more antimicrobial compounds and/or pharmaceutically acceptable ingredients, wherein said peptide or peptidomimetic comprises, essentially consists of, or consists of, preferably comprises, a peptide or peptidomimetic represented by the amino acid sequence of SEQ ID NO: 2, or by an amino acid sequence having at least 60%, 70%, 80%, 85%, 90%, 95%, or 99% identity or similarity with SED ID NO: 2, for the manufacture of a medicament for the treatment, prevention, and/or delaying of an infection on a subject wherein the infection is:
a. a skin infection and/or
b. caused by Staphylococcus aureus.

In an aspect, the invention provides a bacteriocin peptide, or a pharmaceutical composition comprising a bacteriocin peptide, optionally further comprising one or more antimicrobial compounds and/or pharmaceutically acceptable ingredients, wherein said peptide comprises, essentially consists of, or consists of, preferably comprises, a peptide represented by the amino acid sequence of SEQ ID NO: 2, or by an amino acid sequence having at least 60%, 70%, 80%, 85%, 90%, 95%, or 99% identity or similarity with SED ID NO: 2, for use in the treatment, prevention and/or delaying of an infection on a subject wherein the infection is:
a. a skin infection and/or
b. caused by Staphylococcus aureus.

“Inhibition”, “neutralization”, and variations of the terms as used herein have their customary and ordinary meanings as understood by one of skill in the art in view of this disclosure. They include any form of inhibition or arrest of microbial growth and/or division (bacteriostatic effect), as well as any cytotoxic or bactericidal effect (killing). Inhibition and/or neutralization may be full or partial, meaning a whole microbial cell population, such as a target microbial population, or only a part thereof may be growth-inhibited or killed. Partial inhibition may mean that at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 99% of an initial microbial population, such as a target microbial population, is not growth-inhibited or killed.

The ability of a bacteriocin peptide or peptidomimetic to inhibit and/or neutralize a microbial cell, or partially inhibit and/or partially neutralize, (i.e. its biological activity) may be determined using standard methods in the art, for example utilizing standard commercial in vitro tests such as ASTM

E2149-20 or ASTM E1054-08 (ASTM, PA, USA), and the like, as well as methods such as the determination of the formation of an inhibition halo in antimicrobial activity agar plate tests against a microbial cell, such as Staphylococcus aureus and Staphylococcus epidermidis, for example as described in the experimental section herein. As a non-limiting example, a bacteriocin peptide or peptidomimetic as described herein may be considered biologically active against a target microbial cell when application of a solution of said peptide or peptidomimetic with a concentration value of 1000 μg/ml, or about 1000 μg/ml, or lower in antimicrobial activity agar plates tests as described in the experimental section herein results in an inhibition halo being visible. Non-limiting exemplary concentration values may be from 800 to 1000 μg/ml, from 900 to 1000 μg/ml, or from 950 to 1000 μg/ml.

Biological activity of a bacteriocin peptide or peptidomimetic as described herein may also be assessed using its minimum inhibitory concentration (MIC) value against a target microbial cell, preferably a bacterium, more preferably Staphylococcus aureus. “Minimum inhibitory concentration” as used herein refers to the lowest concentration of a bacteriocin peptide or peptidomimetic which inhibits 50% of the growth of the microbial target cell, preferably of a bacterium, more preferably Staphylococcus aureus, after overnight incubation in a liquid culture as compared to a reference culture which has not been exposed to said peptide or peptidomimetic. Alternatively, the minimum bactericidal concentration (MBC), also known as minimum lethal concentration (MLC) may be used to assess biological activity, which refers to the lowest concentration of a bacteriocin peptide or peptidomimetic which is able to kill 99.9% of the cells present in a liquid culture of a bacterium, more preferably of Staphylococcus aureus, after overnight incubation as compared to a reference culture which has not been exposed to said peptide or peptidomimetic. The skilled person understands that a bacteriocin peptide or peptidomimetic exhibiting a lower MIC and/or MBC value relative to another bacteriocin peptide or peptidomimetic may be considered to have an increased biological activity. The skilled person understands that the MIC and/or MBC value of a bacteriocin peptide or peptidomimetic as described herein will vary depending on the target microbial cell. The MIC and/or MBC value of a bacteriocin peptide or peptidomimetic may preferably be determined against Staphylococcus aureus, preferably against Staphylococcus aureus ATCC 6538. Said values may be the same or may differ. The MIC and/or MBC values for different target microbial cells may be determined according to methods commonly used in the art, such as discussed in standard handbooks such as Schwalbe R. et al., Antimicrobial susceptibility testing protocols, Boca Raton: CRC Press (2007) (incorporated herein by reference in its entirety), some of which are also demonstrated in the experimental section herein, and/or commercially available kits such as ETEST® (Biomerieux, NC, USA).

In some embodiments, the MIC and/or MBC value of a bacteriocin peptide or peptidomimetic as described herein ranges from 0.01 to 1000 μg/ml, from 0.1 to 1000 μg/ml, from 1 to 1000 μg/ml, from 2 to 500 μg/ml, from 3 to 125 g/ml, from 4 to 75 μg/ml, from 5 to 25 μg/ml, or from 10 to 20 μg/ml.

In some embodiments, the MIC value of a bacteriocin peptide or peptidomimetic is 0.01 μg/ml or lower, 0.1 μg/ml or lower, 1 μg/ml or lower, 2 μg/ml or lower, 3 μg/ml or lower, 4 μg/ml or lower, 5 μg/ml or lower, 6 μg/ml or lower, 7 μg/ml or lower, 8 μg/ml or lower, 9 g/ml or lower, 10 μg/ml or lower, 11 μg/ml or lower, 12 μg/ml or lower, 13 μg/ml or lower, 14 μg/ml or lower, 15 μg/ml or lower, 16 μg/ml or lower, 17 μg/ml or lower, 18 μg/ml or lower, 19 μg/ml or lower, 20 μg/ml or lower, 21 μg/ml or lower, 22 μg/ml or lower, 23 μg/ml or lower, 24 μg/ml or lower, 25 μg/ml or lower, 26 μg/ml or lower, 27 μg/ml or lower, 28 μg/ml or lower, 29 μg/ml or lower, 30 μg/ml or lower, 31 μg/ml or lower, 32 μg/ml or lower, 33 μg/ml or lower, 34 μg/ml or lower, 35 μg/ml or lower, 36 μg/ml or lower, 37 μg/ml or lower, 38 μg/ml or lower, 39 μg/ml or lower, 40 μg/ml or lower, 41 μg/ml or lower, 42 μg/ml or lower, 43 μg/ml or lower, 44 μg/ml or lower, 45 μg/ml or lower, 46 μg/ml or lower, 47 μg/ml or lower, 48 μg/ml or lower, 49 μg/ml or lower, 50 μg/ml or lower, 51 μg/ml or lower, 52 μg/ml or lower, 53 μg/ml or lower, 54 μg/ml or lower, 55 μg/ml or lower, 56 μg/ml or lower, 57 μg/ml or lower, 58 μg/ml or lower, 59 μg/ml or lower, 60 μg/ml or lower, 61 μg/ml or lower, 62 μg/ml or lower, 63 μg/ml or lower, 64 μg/ml or lower, 65 μg/ml or lower, 66 μg/ml or lower, 67 μg/ml or lower, 68 μg/ml or lower, 69 μg/ml or lower, 70 μg/ml or lower, 71 μg/ml or lower, 72 μg/ml or lower, 73 μg/ml or lower, 74 μg/ml or lower, 75 μg/ml or lower, 76 μg/ml or lower, 77 μg/ml or lower, 78 μg/ml or lower, 79 μg/ml or lower, 80 μg/ml or lower, 81 μg/ml or lower, 82 μg/ml or lower, 83 μg/ml or lower, 84 μg/ml or lower, 85 μg/ml or lower, 86 μg/ml or lower, 87 μg/ml or lower, 88 μg/ml or lower, 89 μg/ml or lower, 90 μg/ml or lower, 91 μg/ml or lower, 92 μg/ml or lower, 93 μg/ml or lower, 94 μg/ml or lower, 95 μg/ml or lower, 96 μg/ml or lower, 97 μg/ml or lower, 98 μg/ml or lower, 99 μg/ml or lower, 100 μg/ml or lower, 110 μg/ml or lower, 120 μg/ml or lower, 130 μg/ml or lower, 140 μg/ml or lower, 150 μg/ml or lower, 160 μg/ml or lower, 170 μg/ml or lower, 180 μg/ml or lower, 190 μg/ml or lower, 200 μg/ml or lower, 250 μg/ml, or lower 300 μg/ml or lower, 400 μg/ml or lower, 500 μg/ml or lower, 550 μg/ml or lower, 600 μg/ml or lower, 650 μg/ml or lower, 700 μg/ml or lower, 750 μg/ml or lower, 800 μg/ml or lower, 850 μg/ml or lower, 900 μg/ml or lower, 950 μg/ml or lower, 1000 μg/ml or lower, preferably it is 16 μg/ml or lower.

In some embodiments, the MBC value of a bacteriocin peptide or peptidomimetic is 0.01 μg/ml or lower, 0.1 μg/ml or lower, 1 μg/ml or lower, 2 μg/ml or lower, 3 μg/ml or lower, 4 μg/ml or lower, 5 μg/ml or lower, 6 μg/ml or lower, 7 g/ml or lower, 8 μg/ml or lower, 9 μg/ml or lower, 10 μg/ml or lower, 11 μg/ml or lower, 12 μg/ml or lower, 13 μg/ml or lower, 14 μg/ml or lower, 15 μg/ml or lower, 16 μg/ml or lower, 17 μg/ml or lower, 18 μg/ml or lower, 19 μg/ml or lower, 20 μg/ml or lower, 21 μg/ml or lower, 22 μg/ml or lower, 23 μg/ml or lower, 24 μg/ml or lower, 25 μg/ml or lower, 26 μg/ml or lower, 27 μg/ml or lower, 28 μg/ml or lower, 29 μg/ml or lower, 30 μg/ml or lower, 31 μg/ml or lower, 32 μg/ml or lower, 33 μg/ml or lower, 34 μg/ml or lower, 35 μg/ml or lower, 36 μg/ml or lower, 37 μg/ml or lower, 38 μg/ml or lower, 39 μg/ml or lower, 40 μg/ml or lower, 41 μg/ml or lower, 42 μg/ml or lower, 43 μg/ml or lower, 44 μg/ml or lower, 45 μg/ml or lower, 46 μg/ml or lower, 47 μg/ml or lower, 48 μg/ml or lower, 49 μg/ml or lower, 50 μg/ml or lower, 51 μg/ml or lower, 52 μg/ml or lower, 53 μg/ml or lower, 54 μg/ml or lower, 55 μg/ml or lower, 56 μg/ml or lower, 57 μg/ml or lower, 58 μg/ml or lower, 59 μg/ml or lower, 60 μg/ml or lower, 61 μg/ml or lower, 62 μg/ml or lower, 63 μg/ml or lower, 64 μg/ml or lower, 65 μg/ml or lower, 66 μg/ml or lower, 67 μg/ml or lower, 68 μg/ml or lower, 69 μg/ml or lower, 70 μg/ml or lower, 71 μg/ml or lower, 72 μg/ml or lower, 73 μg/ml or lower, 74 μg/ml or lower, 75 μg/ml or lower, 76 μg/ml or lower, 77 μg/ml or lower, 78 μg/ml or lower, 79 μg/ml or lower, 80 μg/ml or lower, 81 μg/ml or lower, 82 μg/ml or lower, 83 μg/ml or lower, 84 μg/ml or lower, 85 μg/ml or lower, 86 μg/ml or lower, 87 μg/ml or lower, 88 μg/ml or lower, 89 μg/ml or lower, 90 μg/ml or lower, 91 μg/ml or lower, 92 μg/ml or lower, 93 μg/ml or lower, 94 μg/ml or lower, 95 μg/ml or lower, 96 μg/ml or lower, 97 μg/ml or lower, 98 μg/ml or lower, 99 μg/ml or lower, 100 μg/ml or lower, 110 μg/ml or lower, 120 μg/ml or lower, 130 μg/ml or lower, 140 μg/ml or lower, 150 μg/ml or lower, 160 μg/ml or lower, 170 μg/ml or lower, 180 μg/ml or lower, 190 μg/ml or lower, 200 μg/ml or lower, 250 μg/ml, or lower 300 μg/ml or lower, 400 μg/ml or lower, 500 μg/ml or lower, 550 μg/ml or lower, 600 μg/ml or lower, 650 μg/ml or lower, 700 μg/ml or lower, 750 μg/ml or lower, 800 μg/ml or lower, 850 μg/ml or lower, 900 μg/ml or lower, 950 μg/ml or lower, 1000 μg/ml or lower, preferably it is 16 μg/ml or lower.

Within the context of the invention, a bacteriocin peptide may be produced by a cell or be synthetic. Production of a peptide by a cell can be endogenous or exogenous. Endogenous production refers to production of the peptide by a cell that is natively able to produce it (i.e. a cell that comprises the required genetic information for its production). Preferably, endogenous production refers to production by Bacillus cereus, preferably Bacillus cereus Bc7 (Spanish Type Culture Collection Accession No: CECT 5148). Exogenous production typically refers to production of the peptide by a different organism and/or cell (i.e. a host), by which said peptide is not natively produced, the capability of which having been introduced via means of recombinant DNA technology. Within the context of the invention, the term exogenous production also encompasses cases wherein the native production of the peptide, preferably by Bacillus cereus, more preferably Bacillus cereus Bc7, is increased via means of recombinant DNA technology using standard molecular toolbox techniques as compared to the corresponding endogenous production. Said increase may be achieved by modification of any of the steps of a bacteriocin peptide production, including transcription, post-transcriptional modification, translation, post-translational modification, and secretion. Said increase may be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200% compared to the corresponding endogenous production. A definition of peptide production, alternatively referred to herein as peptide expression, is provided in the section titled “general information”. Exogenous production can be achieved by introduction of a nucleotide sequence comprising a bacteriocin encoding sequence (ORF) to a host organism and/or cell. Recombinant DNA techniques, suitable host organisms and/or cells for exogenous protein production, and culturing methods are well-known in the art and are described in standard handbooks such as Ausubel et al., Current Protocols in Molecular Biology, 3rd edition, John Wiley & Sons Inc (2003) and in Sambrook and Green, Molecular Cloning. A Laboratory Manual, 4th Edition, Cold Spring Harbor Laboratory Press (2012); both of which are incorporated herein by reference in their entireties. To achieve exogenous protein production, a bacteriocin ORF, operably linked to (i.e., in a functional relationship with) a suitable transcription initiation sequence such as a promoter, will typically be introduced to a suitable host cell according to standard techniques. A promoter may be constitutive i.e. allowing constant expression of a bacteriocin peptide, or inducible i.e. only allowing expression of a bacteriocin peptide under specific culture conditions or upon induction with chemical compounds. Optionally, other regulatory sequences such as transcription terminators, enhancers, kozak sequences, polyA sequences and the like may be operably linked to the bacteriocin ORF. The choice of a particular regulatory sequence will depend on the choice of the host cell and is well within the capabilities of the skilled person. As a non-limiting example, a nucleotide sequence comprising, consisting essentially of, or consisting of SEQ ID NO: 55, or a nucleotide sequence having a 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 55, may be introduced in a suitable host cell. The bacteriocin ORF may be stably integrated in a suitable cell's genome or may be introduced in a self-replicating vector. The bacteriocin ORF may be codon-optimized for expression in a particular host cell, e.g. Escherichia coli, using commonly used computer algorithms. A definition of codon optimization is given in the section titled “general information”. Suitable host cells may be selected from mammalian, insect, plant, or microbial cells, preferably are selected from microbial cells. Examples of suitable microbial cells include eukaryotes such as yeasts, filamentous fungi, and algae, and prokaryotes such as bacteria and archaea, of which bacteria is preferred. Bacterial host cells include both Gram-negative and Gram-positive bacteria and can be selected from suitable groups known in the art such as Bacillus species (for example Bacillus cereus, Bacillus anthracis, Bacillus thuringiensis, Bacillus mycoides, Bacillus pseudomycoides, Bacillus cytotoxicus, Bacillus coagulans, Bacillus subtilis, and Bacillus licheniformis), Paenibacillus species, Streptomyces species, Micrococcus species, Corynebacterium species, Acetobacter species, Cyanobacteria species, Salmonella species, Rhodococcus species, Pseudomonas species, Lactobacillus species, Enterococcus species, Alcaligenes species, Klebsiella species, Paenibacillus species, Arthrobacter species, Corynebacterium species, Brevibacterium species, Thermus aquaticus, Pseudomonas stutzeri, Clostridium thermocellus, Escherichia coli, including strains thereof. Non-limiting examples of suitable Bacillus strains are Bc7, ATCC7064, ATCC27877, ATCC12826, BGSC6A3, BGSC6E1, BGSC6E2, BGSC4A9, BGSC4B1, BGSC4C3, HD1, BGSC4E1, BGSC4F1, BGSC4G1, BGSC4H1, BGSC411, BGSC4J1, BGSC4S2, UW85, Soy130, ALF1, ALF9, ALF10, ALF13, ALF19, ALF23, ALF52, ALF53, ALF79, ALF83, ALF85, ALF94, ALF95, ALF98, ALF99, ALF108, ALF109, ALF115, ALF117, ALF133, ALF137, ALF144, ALF154, ALF157, ALF161, ALF166, ALF167, ALF173 LUTZ21, LUTZ58, LUTZ128, SNY14, SNY42, SNY44, SNY45, SNY73, BART8, BAR145, BAR177, MOR1, MOR28, MOR37, SM32, SM43, SM44, VGA19, VGA118, VGA137, VGA562, VGA577, VGA598, AS7-4, AS8-4, AG8-13, AS4-12, ARL8 HS1-3, HS23-11, HS24-8, HS24-9, MS1-9, MS3-2, MS8-2, LS2-2, LS2-12, LS33-2, WS4-12, WS8-8, WS10-15, WS16-4, WS22-12, TNM68, TNM155, TNM243 TG38, TG42, TG 126 DGA34, DGA37, DGA84, DGA94 LN24, LN75, LN100, Z8, and W35, of which strain Bc7 is preferred. Algae host cells may be selected from suitable groups known in the art such as Botryococcus braunii, Chlorella species, Dunaliella tertiolecta, Gracilaria species, Pleurochrysis carterae, and Sargassum species. Yeast host cells may be selected from suitable groups known in the art such as Saccharomyces species (for example, Saccharomyces cerevisiae, Saccharomyces bayanus, Saccharomyces boulardii), Candida species (for example, Candida utilis, Candida krusei), Schizosaccharomyces species (for example Schizosaccharomyces pombe, Schizosaccharomyces japonicus), Pichia or Hansenula species (for example, Pichia pastoris or Hansenula polymorpha) species, and Brettanomyces species (for example, Brettanomyces claussenii). Filamentous fungal host cells may be selected from suitable groups known in the art such as Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocaffimastix, Neurospora, Paecilomyces, Peniciffium, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria. Species include Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, sulphureum, Fusarium sporotrichioides, Fusarium Fusarium torulosum, Fusarium trichothecioides, Fusarium venenaturn, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Penicillium chrysogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride.

A bacteriocin peptide may be isolated and/or purified from its producing cell. Suitable downstream processing methods for isolation and/or purification of products from cell cultures are well-known in the art and are described in standard handbooks such as Wesselingh, J. A and Krijgsman, J., 1st edition, Downstream Processing in Biotechnology, Delft Academic Press (2013), incorporated herein by reference in its entirety. Examples of suitable isolation and/or purification techniques are chromatographic methods such as high performance liquid chromatography, size exclusion chromatography, ion exchange chromatography, affinity chromatography, immunoaffinity chromatography, immunoprecipitation via the use of tags, and the like. Accordingly, in some embodiments the bacteriocin peptide is an isolated and/or purified peptide.

Within the context of the invention, a bacteriocin peptide may be produced in vitro, using isolated and/or purified cellular components (cell-free extracts) comprising the necessary transcription and translation machinery. In vitro protein production typically comprises transcription and translation of isolated circular or linear DNA, or only translation when isolated mRNA is used as a template, said DNA or mRNA comprising a bacteriocin encoding sequence optionally operably linked to regulatory sequences as discussed elsewhere herein. The corresponding cellular components may be isolated/purified and the reaction conditions can be chosen according to standard methods, such as for example described in Gregorio et al., Methods Protoc 2(1):24 (2019), incorporated herein by reference in its entirety. Alternatively, commercial in vitro protein synthesis kits such as PURExpress® (New England Biolabs, MA, USA) may be used. Depending the on the cell used for isolation/purification of the cellular components, e.g. Escherichia coli, and/or the commercial kit used, the bacteriocin ORF may be codon optimized for expression in that particular cell and/or commercial kit. As a non-limiting example, a nucleotide sequence comprising, consisting essentially of, or consisting of, preferably comprising, SEQ ID NO: 55, or a nucleotide sequence having a 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 55, may be used in conjunction with PURExpress® according to the manufacturer's protocol to produce a bacteriocin. Accordingly, in some embodiments the bacteriocin peptide is an in vitro produced peptide.

Within the context of the invention, a bacteriocin peptide may be synthetic. The term “synthetic peptide” has its customary and ordinary meaning as understood by one of skill in the art in view of this disclosure. It refers to a peptide which is generated by means of chemical peptide synthesis. A synthetic bacteriocin peptide according to the invention may be prepared or synthesized using conventional methods that are well-known in the art. For instance, peptides can be synthesized by commonly used solid-phase synthesis methods such as those that involve a tert-butyloxycarbonyl-protecting group (t-BOC) or fluorenylmethyloxycarbonyl-protecting group (FMOC) for protection of alpha-amino groups. In such methods, amino acids are added sequentially to a growing amino acid chain. Such methods are, for instance, described in Merrifield, J. Am. Chem. Soc. 85(14): 2149-2154 (1963), and Atherton & Sheppard, Solid Phase Peptide Synthesis: A practical Approach, IRL Press, Oxford, UK (1999), both of which are incorporated herein by reference in their entireties. Accordingly, in some embodiments, the bacteriocin peptide is a synthetic peptide.

Within the context of the invention, a bacteriocin may be a peptidomimetic. As used herein, a “peptidomimetic” (alternatively referred to as “mimetic”) is understood to encompass all compounds whose essential elements mimic a natural peptide and which retain the ability to interact with the biological target and exert the natural peptide's biological activity. The biological activity of a bacteriocin peptidomimetic may be the same, decreased, or increased as compared to a bacteriocin peptide. Decreased biological activity of a bacteriocin peptidomimetic may mean that the peptidomimetic exhibits at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the biological activity of the corresponding bacteriocin peptide. Increased biological activity of a bacteriocin peptidomimetic may mean that the peptidomimetic exhibits an increase of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200% in biological activity as compared to the corresponding bacteriocin peptide. A definition of “biological activity” and measurement methods thereof are provided elsewhere herein.

In some embodiments, the peptidomimetic comprises, consists essentially of, or consists of, preferably comprises, a non-naturally occurring amino acid sequence. In some embodiments, the peptidomimetic does not occur in nature and is considered to be man-made. Peptidomimetics typically arise either from modification of an existing peptide, or by designing similar systems that mimic peptides, such as peptoids and β-peptides. Structures and synthesis of peptidomimetics are for instance described in William D. Lubell (ed.), Peptidomimetics I and II, Topics in Heterocyclic Chemistry (Book 48), Springer 1st ed., XVI, 310 p (2017); Trabocchi A. Chapter 6—Principles and applications of small molecule peptidomimetics, Small Molecule Drug Discovery Methods, Molecules and Applications, pp 163-195, Elsevier (2020); Vagner et al., Curr Opin Chem Biol. 12(3): 292-296 (2008), all of which incorporated herein by reference in their entireties. A bacteriocin peptidomimetic may be a structural mimetic of a bacteriocin peptide described herein. Structural mimetics, also known as type I mimetics, have analogous structural features to the bacteriocin peptide they mimic. A bacteriocin peptidomimetic may be a functional mimetic of a bacteriocin peptide described herein. Functional mimetics, also known as type II mimetics, retain the ability to interact with the biological target and exert the natural peptide's biological activity without apparent structural analogy to the peptide. A bacteriocin peptidomimetic may be a functional-structural mimetic of a bacteriocin peptide described herein. Functional-structural mimetics, also known as type III mimetics, generally comprise a scaffold having a structure different from the bacteriocin peptide that they mimic, in which all the functional groups needed for the biological activity are mounted in a well-defined spatial orientation.

In some embodiments, a bacteriocin peptidomimetic corresponds to a bacteriocin peptide in which a modification has been introduced, for example to the backbone and/or the side chains.

In some embodiments, a bacteriocin peptidomimetic corresponds to a bacteriocin peptide in which a non-natural amino acid has been introduced. Examples of non-natural amino acids are provided later herein. In some embodiments, a natural amino acid is substituted by a non-natural amino acid or a D-amino acid, which may, for example, be corresponding as described later herein.

In some embodiments, a bacteriocin peptidomimetic corresponds to a bacteriocin peptide in which the peptide backbone has been replaced completely, for example by a heterocycle, a sugar, or other scaffold. Examples of suitable scaffolds are known to the skilled person and discussed, for example, in Pelay-Gimeno et al., Angew Chem Int Ed Engl; 54(31): 8896-8927 (2015), incorporated herein by reference in its entirety. In some embodiments, a bacteriocin peptidomimetic corresponds to a peptoid. In some embodiments, a bacteriocin peptidomimetic corresponds to a β-peptide.

Modification of an existing peptide may be the result of natural processes, such as post-translational processing, or chemical modification techniques. In some embodiments, a peptidomimetic refers to a compound containing non-peptidic structural elements. Typical but non-limiting examples of non-peptidic structural elements are modifications of one or more existing amino acids, conformational restraints, cyclization of the polypeptide, isosteric replacement or other modifications. In some embodiments, a peptidomimetic may contain one or more or all substitutions of an amino acid by the corresponding D-amino acid. As used herein, “corresponding D-amino acid” denotes the D-amino acid counterpart of an L-amino acid. In some embodiments, a peptidomimetic may also contain non-natural amino acids. As used herein, “non-natural amino acid” has its customary and ordinary meaning as understood by one of skill in the art in view of this disclosure. It refers to non-genetically encoded amino acids, irrespective of whether they appear in nature or not. Non-natural amino acids that can be present in a peptidomimetic as described herein include: —amino acids; p-acyl-L-phenylalanine; N-acetyl lysine; O-4-allyl-L-tyrosine; 2-aminoadipic acid; 3-aminoadipic acid; beta-alanine; 4-tert-butyl hydrogen 2-azidosuccinate; beta-aminopropionic acid; 2-aminobutyric acid; 4-aminobutyric acid; 2,4-diamino butyric acid; 6-aminocaproic acid; 2-aminoheptanoic acid; 2-aminoisobutyric acid; 3-aminoisobutyric acid; 2-aminopimelic acid; p-aminophenylalanine; 2,3-diaminobutyric acid; 2,3-diamino propionic acid; 2,2′-diaminopimelic acid; p-amino-L-phenylalanine; p-azido-L-phenylalanine; D-allyl glycine; p-benzoyl-L-phenylalanine; 3-benzothienyl alanine p-bromophenylalanine; t-butylalanine; t-butylglycine; 4-chlorophenylalanine; cyclohexylalanine; cysteic acid; D-citrulline; thio-L-citrulline; desmosine; epsilon-amino hexanoic acid; N-ethylglycine; N-ethylasparagine; 2-fluorophenylalanine; 3-fluorophenylalanine; 4-fluorophenylalanine; homoarginine; homocysteine; homoserine; hydroxy lysine; alio-hydroxy lysine; 3-(3-methyl-4-nitrobenzyl)-L-histidine methyl ester; isodesmosine; allo-isoleucine; isopropyl-L-phenylalanine; 3-methyl-phenylalanine; N-methylglycine; N-methylisoleucine; 6-N-methyllysine; O-methyl-L-tyrosine; N-methylvaline; methionin sulfoxide; 2-napthylalanine; L-3-(2-naphthyl)alanine; isoserine; 3-phenylserine; norvaline; norleucine; 5,5,5-trifluoro-DL-leucine; ornithine; 3-chloro-tyrosine; N5-carbamoylornithine; penicillamine; phenylglycine; piperidinic acid; pyridylalanine; 1,2,3,4-tetrahydro-isoquinoline-3-carboxylic acid; beta-2-thienylalanine; Y-carboxy-DL-glutamic acid; 4-fluoro-DL-glutamic acid; D-thyroxine; allo-threonine; 5-hydroxy-tryptophan; 5-methoxy-tryptophan; 5-fluoro-tryptophan; 3-fluoro-valine. In some embodiments, a natural amino acid of a bacteriocin peptide or peptidomimetic according to the invention is substituted by a corresponding non-natural amino acid. As used herein, a “corresponding non-natural amino acid” refers to a non-natural amino acid that is a derivative of the reference natural amino acid. For instance, a natural amino acid can be substituted by the corresponding β-amino acid, which has its amino group bonded to the β-carbon rather than the α-carbon. In some embodiments, a peptide or peptidomimetic of the invention may further be provided with a targeting moiety. It is known that peptidomimetics are able to circumvent some of the disadvantages associated with natural peptides: e.g. stability against proteolysis (duration of activity) and poor bioavailability. Certain other properties, such as receptor selectivity or potency, often can be substantially improved.

Within the context of the invention, a bacteriocin peptide or peptidomimetic may further be modified by natural processes, such as post-translational processing, or by chemical modification techniques. Such modifications may be inserted in the peptide at any location, including in the backbone, amino acid side-chains and at the N- or C-terminus. Multiple types of modifications may occur in a single peptide, or a peptide may comprise several modifications of a single type. Types of modifications and modification techniques are well-known in the art and described in standard handbooks such as Peptide Modifications to Increase Metabolic Stability and Activity, 1st edition, Ed. Predrag Cudic, Humana Press (2013), incorporated herein by reference in its entirety. Accordingly, in some embodiments, the bacteriocin peptide or peptidomimetic comprises at least one amino acid modification selected from the group consisting of alkylation, acetylation, amidation, acylation, phosphorylation, methylation, demethylation, ADP-ribosylation, disulfide bond formation, ubiquitination, gamma-carboxylation, glycosylation, hydroxylation, iodination, oxidation, pegylation, succinylation, and sulfation, preferably selected from methylation or glycosylation.

Within the context of the invention, a bacteriocin peptide or peptidomimetic may comprise one or more modifications in its sequence, resulting in bacteriocin peptide or peptidomimetic mutants (alternatively referred to herein as variants). Said sequence modifications may include amino acid substitutions, deletions and/or insertions. Mutant peptides or peptidomimetics can, for example, be synthetically made or made by cellular (or in vitro) production as described elsewhere herein, after modifying the nucleotide sequence encoding for said peptides using mutagenesis techniques known to the skilled person, such as, random mutagenesis, site-directed mutagenesis, directed evolution, gene shuffling, CRISPR/Cas-mediated mutagenesis and the like, so that the resulting nucleotide sequence encodes a peptide that differs by at least one amino acid from the non-modified peptide or peptidomimetic, i.e. wherein at least one amino acid is substituted by a different amino acid and/or at least one amino acid is deleted and/or at least one amino acid is inserted. Amino acid substitutions may be conservative. A definition of a “conservative” substitution is provided in the section titled “general information”. Mutant peptides and/or peptidomimetics according to the invention may retain decreased, but still detectable, or increased biological activity as compared to the corresponding non-modified peptide or peptidomimetic. Biological activity (i.e. antimicrobial activity) against a target microbial cell, preferably a bacterium, more preferably Staphylococcus aureus, may be assessed as described elsewhere herein. Decreased biological activity of a mutant may mean that the mutant exhibits at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the biological activity of the corresponding non-modified bacteriocin peptide or peptidomimetic. Increased biological activity of a mutant may mean that the mutant exhibits an increase of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200% in biological activity as compared to the corresponding non-modified bacteriocin peptide or peptidomimetic.

Within the context of the invention, the physicochemical properties of a bacteriocin peptide or peptidomimetic may be the same or differ as compared to a corresponding naturally-occurring (wild-type) peptide. The skilled person is aware of such properties, non-limiting examples of which include susceptibility to enzymatic degradation (e.g. by proteinases, peptidases, aminopeptidases, carboxypeptidases, RNases, phospholipases, amylases, and the like), susceptibility to degradation by organic solvents (e.g. to acetone, chloroform, acetonitrile, ethanol, 2-propanol, butanol, methanol, and the like), susceptibility to degradation by surfactants (e.g. anionic, non-ionic, cationic, amphoteric, silicon-based, fluorinated, polymeric, and the like), susceptibility to reducing agents (e.g. DTT, β-mercaptoethanol, and the like), susceptibility to heat degradation, pH optimum, and the like. In some embodiments, a bacteriocin peptide or peptidomimetic exhibits at least one improved physicochemical property as compared to a corresponding naturally-occurring (wild-type) peptide. Physicochemical properties of peptides or peptidomimetics may be assessed by commonly used methods in the art, such as discussed in standard handbooks like Hansen, P. R., Antimicrobial Peptides: Methods and Protocols, 1^{st} Edition, Humana Press, US, (2017) and Remington: The Science and Practice of Pharmacy, 23^{rd} ed., Ed. Adejare A., Academic Press, US (2021), both of which are incorporated herein by reference in their entireties.

In some embodiments, a bacteriocin peptide of peptidomimetic comprises, essentially consists of, or consists of, preferably comprises, a peptide or peptidomimetic represented by a cerein 7B sequence or mutant thereof. In some embodiments, a bacteriocin peptide or peptidomimetic sequence comprises, essentially consists of, or consists of, preferably comprises, a peptide or peptidomimetic represented by the amino acid sequence of SEQ ID NO: 1 or 2, preferably SEQ ID NO: 2, or by an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%, preferably at least 80%, 85%, 90%, 95%, or 99%, identity or similarity with SEQ ID NO: 1 or 2, preferably with SEQ ID NO: 2.

Standard protocols in the art such as alanine scanning may be used to determine the contribution of a specific residue to the stability or function of a given peptide or peptidomimetic. Alanine scanning is commonly used because alanine has a non-bulky, chemically inert, methyl functional group that nevertheless mimics the secondary structure preferences that many of the other amino acids possess. Alternative techniques such as valine or leucine scanning may also be used. As described in the experimental section herein, the present inventors have generated a bacteriocin peptide mutant library utilizing alanine scanning to determine the contribution of specific amino acid residues of SEQ ID NO: 2 to its activity against microbial target cells, using Staphylococcus aureus as a case study. The skilled person understands that even if a particular amino acid of SEQ ID NO: 2 is not replaceable by an alanine, it may still be replaceable by other amino acids. As a non-limiting example, an amino acid may be replaceable by amino acids having side chains with similar properties (conservative substitutions), such as the replacement of a lysine by a histidine. A definition of a “conservative” substitution is provided in the section titled “general information”.

Utilizing alanine scanning, the inventors have found that biological activity may be maintained in a peptide or peptidomimetic comprising the amino acid substitutions represented by the following amino acid consensus sequence:
GWWXaaXaaWGKCVXaaGXaaIGXaaXaaXaaTGXaaLXaaGXaaXaaXaaGSXaaVPVIGTXaa IGXaaXaaIGXaaVXaaGXaaLTXaaXaaXaaXaaFC (SEQ ID NO: 3), wherein Xaa represents any amino acid.

“Any amino acid” as used herein includes any of the natural (L- and D-configuration) amino acids, non-natural aminoacids, as well as modified versions of natural and/or non-natural aminoacids, as described elsewhere herein.

Accordingly, in some embodiments a bacteriocin peptide or peptidomimetic sequence comprises, essentially consists of, or consists of, preferably comprises, a peptide or peptidomimetic represented by the amino acid sequence of SEQ ID NO: 3.

A bacteriocin peptide or peptidomimetic may comprise, essentially consist of, or consist of, preferably comprise, a peptide or peptidomimetic represented by an amino acid sequence wherein at least one amino acid of SEQ ID NO: 2 has been substituted by any amino acid. Said substitution may be conservative. Said substitution may correspond to specific amino acid positions of SEQ ID NO: 2. When multiple amino acids are substituted, they may correspond to consecutive positions or may be spatially apart in the peptide sequence. Determination of specific amino acids to be substituted in a peptide sequence corresponding to specific positions of SEQ ID NO: 2 may be performed by routine sequence alignment methods, further elaborated upon in the section titled “general information” herein. The skilled person understands that the glycine (G) residue at the N-terminus end of SEQ ID NO: 2 corresponds to position 1, that the cysteine (C) at the C-terminus end of SEQ ID NO: 2 corresponds to position 56, and that the amino acids in between the two ends of SEQ ID NO: 2 correspond to positions 2-55, respectively.

Accordingly, in some embodiments, a bacteriocin peptide or peptidomimetic sequence comprises, essentially consists of, or consists of, preferably comprises, a peptide or peptidomimetic represented by an amino acid sequence wherein at least one amino acid, at least two amino acids, at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids, at least sixteen amino acids, at least seventeen amino acids, at least eighteen amino acids, at least nineteen amino acids, at least twenty amino acids, at least twenty-one amino acids, at least twenty-two amino acids, or at least twenty-three amino acids, preferably at least one amino acid, of SEQ ID NO: 2 have been substituted by any amino acid. In some embodiments, at least two amino acids of SEQ ID NO: 2 have been substituted by any amino acid. In some embodiments, at least three amino acids of SEQ ID NO: 2 have been substituted by any amino acid. In some embodiments, at least four amino acids of SEQ ID NO: 2 have been substituted by any amino acid. In some embodiments, at least five amino acids of SEQ ID NO: 2 have been substituted by any amino acid. In some embodiments, at least six amino acids of SEQ ID NO: 2 have been substituted by any amino acid. In some embodiments, at least seven amino acids of SEQ ID NO: 2 have been substituted by any amino acid. In some embodiments, at least eight amino acids of SEQ ID NO: 2 have been substituted by any amino acid. In some embodiments, at least nine amino acids of SEQ ID NO: 2 have been substituted by any amino acid. In some embodiments, at least ten amino acids of SEQ ID NO: 2 have been substituted by any amino acid. In some embodiments, at least eleven amino acids of SEQ ID NO: 2 have been substituted by any amino acid. In some embodiments, at least twelve amino acids of SEQ ID NO: 2 have been substituted by any amino acid. In some embodiments, at least thirteen amino acids of SEQ ID NO: 2 have been substituted by any amino acid. In some embodiments, at least fourteen amino acids of SEQ ID NO: 2 have been substituted by any amino acid. In some embodiments, at least fifteen amino acids of SEQ ID NO: 2 have been substituted by any amino acid. In some embodiments, at least sixteen amino acids of SEQ ID NO: 2 have been substituted by any amino acid. In some embodiments, at least seventeen amino acids of SEQ ID NO: 2 have been substituted by any amino acid. In some embodiments, at least eighteen amino acids of SEQ ID NO: 2 have been substituted by any amino acid. In some embodiments, at least nineteen amino acids of SEQ ID NO: 2 have been substituted by any amino acid. In some embodiments, at least twenty amino acids of SEQ ID NO: 2 have been substituted by any amino acid. In some embodiments, at least twenty-one amino acids of SEQ ID NO: 2 have been substituted by any amino acid. In some embodiments, at least twenty-two amino acids of SEQ ID NO: 2 have been substituted by any amino acid. In some embodiments, at least twenty-three amino acids of SEQ ID NO: 2 have been substituted by any amino acid. Preferred positions for substitutions may be selected from the group of positions 4, 5, 11, 13, 16, 17, 18, 21, 23, 25, 26, 27, 30, 37, 40, 41, 44, 46, 48, 51, 52, 53, or 54 of SEQ ID NO: 2.

In some embodiments, a bacteriocin peptide or peptidomimetic sequence comprises, essentially consists of, or consists of, preferably comprises, a peptide or peptidomimetic represented by an amino acid sequence wherein the asparagine (N) corresponding to position 4 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the serine (S) corresponding to position 5 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic wherein the alanine (A) corresponding to position 11 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the threonine (T) corresponding to position 13 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the glycine (G) corresponding to position 16 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the alanine (A) corresponding to position 17 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the glycine (G) corresponding to position 18 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the glycine (G) corresponding to position 21 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the glycine (G) corresponding to position 23 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the alanine (A) corresponding to position 25 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the alanine (A) corresponding to position 26 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the alanine (A) corresponding to position 27 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the alanine (A) corresponding to position 30 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the glycine (G) corresponding to position 37 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the glycine (G) corresponding to position 40 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the alanine (A) corresponding to position 41 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the glycine (G) corresponding to position 44 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the serine (S) corresponding to position 46 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the glycine (G) corresponding to position 48 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the glycine (G) corresponding to position 51 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the alanine (A) corresponding to position 52 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the alanine (A) corresponding to position 53 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein the threonine (T) corresponding to position 54 of SEQ ID NO: 2 has been substituted by any amino acid.

In some embodiments, a bacteriocin peptide or peptidomimetic sequence comprises, essentially consists of, or consists of, preferably comprises, a peptide or peptidomimetic represented by an amino acid sequence wherein at least one glycine (G) of SEQ ID NO: 2 has been substituted by an amino acid selected from alanine (A), valine (V), leucine (L), and isoleucine (I).

In some embodiments, a bacteriocin peptide or peptidomimetic sequence comprises, essentially consists of, or consists of, preferably comprises, a peptide or peptidomimetic represented by an amino acid sequence wherein at least one tryptophan (W) of SEQ ID NO: 2 has been substituted by a phenylalanine (F) or tyrosine (Y).

In some embodiments, a bacteriocin peptide or peptidomimetic sequence comprises, essentially consists of, or consists of, preferably comprises, a peptide or peptidomimetic represented by an amino acid sequence wherein at least one asparagine (N) of SEQ ID NO: 2 has been substituted by an amino acid selected from serine (S), threonine (T), and glutamine (Q).

In some embodiments, a bacteriocin peptide or peptidomimetic sequence comprises, essentially consists of, or consists of, preferably comprises, a peptide or peptidomimetic represented by an amino acid sequence wherein at least one serine (S) of SEQ ID NO: 2 has been substituted by an amino acid selected from asparagine (N), threonine (T), and glutamine (Q).

In some embodiments, a bacteriocin peptide or peptidomimetic sequence comprises, essentially consists of, or consists of, preferably comprises, a peptide or peptidomimetic represented by an amino acid sequence wherein at least one lysine (K) of SEQ ID NO: 2 has been substituted by an arginine (R) or a histidine (H).

In some embodiments, a bacteriocin peptide or peptidomimetic sequence comprises, essentially consists of, or consists of, preferably comprises, a peptide or peptidomimetic represented by an amino acid sequence wherein at least one cysteine (C) of SEQ ID NO: 2 has been substituted by an amino acid selected from methionine (M), selenocysteine (U), and proline (P).

In some embodiments, a bacteriocin peptide or peptidomimetic sequence comprises, essentially consists of, or consists of, preferably comprises, a peptide or peptidomimetic represented by an amino acid sequence wherein at least one valine (V) of SEQ ID NO: 2 has been substituted by an amino acid selected from alanine (A), glycine (G), leucine (L), and isoleucine (I).

In some embodiments, a bacteriocin peptide or peptidomimetic sequence comprises, essentially consists of, or consists of, preferably comprises, a peptide or peptidomimetic represented by an amino acid sequence wherein at least one alanine (A) of SEQ ID NO: 2 has been substituted by an amino acid selected from valine (V), glycine (G), leucine (L), and isoleucine (I).

In some embodiments, a bacteriocin peptide or peptidomimetic sequence comprises, essentially consists of, or consists of, preferably comprises, a peptide or peptidomimetic represented by an amino acid sequence wherein at least one threonine (T) of SEQ ID NO: 2 has been substituted by an amino acid selected from serine (S), asparagine (N), and glutamine (Q).

In some embodiments, a bacteriocin peptide or peptidomimetic sequence comprises, essentially consists of, or consists of, preferably comprises, a peptide or peptidomimetic represented by an amino acid sequence wherein at least one isoleucine (I) of SEQ ID NO: 2 has been substituted by an amino acid selected from valine (V), alanine (A), glycine (G), and leucine (L).

In some embodiments, a bacteriocin peptide or peptidomimetic sequence comprises, essentially consists of, or consists of, preferably comprises, a peptide or peptidomimetic represented by an amino acid sequence wherein at least one leucine (L) of SEQ ID NO: 2 has been substituted by an amino acid selected from valine (V), alanine (A), glycine (G), and isoleucine (I).

In some embodiments, a bacteriocin peptide or peptidomimetic sequence comprises, essentially consists of, or consists of, preferably comprises, a peptide or peptidomimetic represented by an amino acid sequence wherein at least one proline (P) of SEQ ID NO: 2 has been substituted by a methionine (M) or cysteine (C).

In some embodiments, a bacteriocin peptide or peptidomimetic sequence comprises, essentially consists of, or consists of, preferably comprises, a peptide or peptidomimetic represented by an amino acid sequence wherein at least one phenylalanine (F) of SEQ ID NO: 2 has been substituted by a tyrosine (Y) or tryptophan (W).

In some embodiments, a bacteriocin peptide or peptidomimetic sequence comprises, essentially consists of, or consists of, preferably comprises, a peptide or peptidomimetic represented by an amino acid sequence wherein the amino acid sequence of SEQ ID NO: 2 or 3, preferably SEQ ID NO: 2, has been modified in length. Said length modification may arise from the deletion of amino acids (shortening) and/or insertion of amino acids (lengthening). An inserted amino acid can be any amino acid as discussed elsewhere herein. The skilled person understands that deletion and/or insertion of amino acids may occur at any position of the peptide chain, including the end points and any position in between. In some embodiments, a bacteriocin peptide or peptidomimetic sequence comprises, essentially consists of, or consists of, preferably comprises, a peptide or peptidomimetic represented by an amino acid sequence wherein the amino acid sequence of SEQ ID NO: 2 or 3, preferably SEQ ID NO: 2, has been shortened by at least one amino acid, at least two amino acids, at least three amino acids, at least four amino acids, at least five amino acids, at least six amino acids, at least seven amino acids, at least eight amino acids, at least nine amino acids, or at least ten amino acids, preferably by at least one amino acid. In some embodiments, a bacteriocin peptide or peptidomimetic sequence comprises, essentially consists of, or consists of, preferably comprises, a peptide or peptidomimetic represented by an amino acid sequence wherein the amino acid sequence of SEQ ID NO: 2 or 3, preferably SEQ ID NO: 2, has been lengthened by at least one amino acid, at least two amino acids, at least three amino acids, at least four amino acids, at least five amino acids, at least six amino acids, at least seven amino acids, at least eight amino acids, at least nine amino acids, or at least ten amino acids, preferably by at least one amino acid.

In some embodiments, a bacteriocin peptide or peptidomimetic sequence comprises, essentially consists of, or consists of, preferably comprises, a peptide or peptidomimetic represented by an amino acid sequence wherein the amino acid sequence of SEQ ID NO: 2 or 3, preferably SEQ ID NO: 2, has been shortened by at least one amino acid at its N-terminus end.

In some embodiments, a bacteriocin peptide or peptidomimetic sequence comprises, essentially consists of, or consists of, preferably comprises, a peptide or peptidomimetic represented by an amino acid sequence wherein the amino acid sequence of SEQ ID NO: 2 or 3, preferably SEQ ID NO: 2, has been shortened by at least one amino acid at its C-terminus end.

In some embodiments, a bacteriocin peptide or peptidomimetic sequence comprises, essentially consists of, or consists of, preferably comprises, a peptide or peptidomimetic represented by an amino acid sequence wherein the amino acid sequence of SEQ ID NO: 2 or 3, preferably SEQ ID NO: 2, has been lengthened by addition of at least one or at least two amino acids to its N-terminus end.

In some embodiments, a bacteriocin peptide or peptidomimetic sequence comprises, essentially consists of, or consists of, preferably comprises, a peptide or peptidomimetic represented by an amino acid sequence wherein the amino acid sequence of SEQ ID NO: 2 or 3, preferably SEQ ID NO: 2, has been lengthened by addition of at least one or at least two amino acids to its C-terminus end.

In some embodiments, a bacteriocin peptide or peptidomimetic sequence comprises, essentially consists of, or consists of, preferably comprises, a peptide or peptidomimetic represented by an amino acid sequence wherein the amino acid sequence of SEQ ID NO: 2 or 3, preferably SEQ ID NO: 2, has been lengthened by the addition of a tryptophan to its C-terminus end.

Non-limiting exemplary amino acid sequences representing peptides or peptidomimetics that a peptide or peptidomimetic according to the invention may comprise, essentially consist of, or consist of, preferably comprise, are given in Table 1:

TABLE 1
Exemplary amino acid sequences

| SEQ ID | | |
|---|---|---|
| NO | Name | Sequence |
| 2 | cerein 7B | GWWNSWGKCVAGTIGGAGTGGLGGAAAGSAVPVIGTGIGGAIGGVSGGL |
| | | TGAATFC |
| 3 | bacteriocin | GWWXaaXaaWGKCVXaaGXaalGXaaXaaXaaTGXaaLXaaGXaaXaaXaaGS |
| | consensus | XaaVPVIGTXaaIGXaaXaaIGXaaVXaaGXaaLTXaaXaaXaaXaaFC |
| 7 | mutant 4 | GWWASWGKCVAGTIGGAGTGGLGGAAAGSAVPVIGTGIGGAIGGVSGGL |
| | (A5) | TGAATFC |
| 8 | mutant 5 | GWWNAWGKCVAGTIGGAGTGGLGGAAAGSAVPVIGTGIGGAIGGVSGGL |
| | (A6) | TGAATFC |
| 15 | mutant 12 | GWWNSWGKCVAGAIGGAGTGGLGGAAAGSAVPVIGTGIGGAIGGVSGGL |
| | (B1) | TGAATFC |
| 18 | mutant 15 | GWWNSWGKCVAGTIGAAGTGGLGGAAAGSAVPVIGTGIGGAIGGVSGGL |
| | (B4) | TGAATFC |
| 19 | mutant 16 | GWWNSWGKCVAGTIGGAATGGLGGAAAGSAVPVIGTGIGGAIGGVSGGL |
| | (B5) | TGAATFC |
| 22 | mutant 19 | GWWNSWGKCVAGTIGGAGTGALGGAAAGSAVPVIGTGIGGAIGGVSGGL |
| | (B8 | TGAATFC |
| 24 | mutant 21 | GWWNSWGKCVAGTIGGAGTGGLAGAAAGSAVPVIGTGIGGAIGGVSGGL |
| | (B10) | TGAATFC |
| 34 | mutant 31 | GWWNSWGKCVAGTIGGAGTGGLGGAAAGSAVPVIGTAIGGAIGGVSGGL |
| | (C8) | TGAATFC |
| 37 | mutant 34 | GWWNSWGKCVAGTIGGAGTGGLGGAAAGSAVPVIGTGIGAAIGGVSGGL |
| | (C11) | TGAATFC |
| 40 | mutant 37 | GWWNSWGKCVAGTIGGAGTGGLGGAAAGSAVPVIGTGIGGAIGAVSGGL |
| | (D2) | TGAATFC |
| 42 | mutant 39 | GWWNSWGKCVAGTIGGAGTGGLGGAAAGSAVPVIGTGIGGAIGGVAGGL |
| | (D4) | TGAATFC |
| 44 | mutant 41 | GWWNSWGKCVAGTIGGAGTGGLGGAAAGSAVPVIGTGIGGAIGGVSGAL |
| | (D6) | TGAATFC |
| 47 | mutant 44 | GWWNSWGKCVAGTIGGAGTGGLGGAAAGSAVPVIGTGIGGAIGGVSGGL |
| | (D9) | TAAATFC |
| 48 | mutant 45 | GWWNSWGKCVAGTIGGAGTGGLGGAAAGSAVPVIGTGIGGAIGGVSGGL |
| | (D10) | TGAAAFC |
| 51 | mutant 48 | GWWNSWGKCVAGTIGGAGTGGLGGAAAGSAVPVIGTGIGGAIGGVSGGL |
| | (E2) | TGAATFCW |

In some embodiments a bacteriocin peptide or peptidomimetic comprises, essentially consists of, or consists of, preferably comprises, a peptide or peptidomimetic represented by the amino acid sequence of any one of SEQ ID NOS: 2, 3, 7, 8, 15, 18, 19, 22, 24, 34, 37, 40, 42, 44, 47, 48, 51, 52, 53, or 54, preferably of any one of SEQ ID NOS: 2, 3, 7, 8, 15, 18, 19, 22, 24, 34, 37, 40, 42, 44, 47, 48, or 51, or by an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with any one of SEQ ID NOs: 2, 3, 7, 8, 15, 18, 19, 22, 24, 34, 37, 40, 42, 44, 47, 48, 51, 52, 53, or 54, preferably with any one of SEQ ID NOs: 2, 3, 7, 8, 15, 18, 19, 22, 24, 34, 37, 40, 42, 44, 47, 48, or 51.

In some embodiments a bacteriocin peptide or peptidomimetic comprises, essentially consists of, or consists of, preferably comprises, a peptide or peptidomimetic represented by the amino acid sequence of any one of SEQ ID NOs: 7, 8, 15, 18, 19, 22, 24, 34, 37, 40, 42, 44, 47, 48, or 51, or by an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with any one of SEQ ID NOs: 7, 8, 15, 18, 19, 22, 24, 34, 37, 40, 42, 44, 47, 48, or 51.

In some embodiments, a bacteriocin peptide or peptidomimetic does not comprise a peptide or peptidomimetic represented by the contiguous amino acid sequence GWGDVL (SEQ ID NO: 56) as the first 6 amino acids of its N-terminus end. In some embodiments, a bacteriocin peptide or peptidomimetic does not comprise a peptide or peptidomimetic represented by the contiguous amino acid sequence GWGDVL (SEQ ID NO: 56). In some embodiments, a bacteriocin peptide or peptidomimetic does not comprise the contiguous amino acid sequence GWGDVL (SEQ ID NO: 56).

Within the context of the invention, a bacteriocin peptide or peptidomimetic may be in salt form. Suitable salts forms of peptides and peptidomimetics and their preparation are known in the art and discussed in standard handbooks, such as Remington: The Science and Practice of Pharmacy (supra) and Koutsopoulos, Peptide Applications in Biomedicine, Biotechnology and Bioengineering, 1st Edition, Woodhead Publishing, UK (2017), incorporated herein by reference in their entireties. Preparation of peptide salts generally involves mixing of the peptide or peptidomimetic with an acid or base, for instance, by reacting the free acid or free base forms of the peptide or peptidomimetic with one or more equivalents of the appropriate acid or base in a solvent or medium in which the salt is insoluble, or in a solvent such as water, which is then removed by vacuum or by freeze-drying, or by exchanging the cations of an existing salt for another cation on a suitable ion exchange resin.

The term “infection” as used herein has its customary and ordinary meaning as understood by one of skill in the art in view of this disclosure. It refers to the invasion and growth of a microbial cell in a subject's (host's) body and/or tissue, which may be accompanied by spreading throughout the body and/or to other tissues. An infection may result in injury to the infected body and/or tissue. An infection may result in a disease. Said microbial cell may come into contact with the subject and/or tissue via the environment, such as physical contact with a contaminated surface, or may already be present in the subject and/or tissue as part of the microbiome of said tissue and/or subject. An infection may be caused by a pathogenic or potentially pathogenic microbial cell. “Pathogenicity” as used herein has its customary and ordinary meaning as understood by one of skill in the art in view of this disclosure. It refers to the infection-causing capacity of a microbial cell. A potentially pathogenic microbial cell will cause an infection under certain conditions, for example in cases wherein the immune system of the host is compromised or a bodily wound allows for entry of said cell in the body. A preferred infection may be caused by a bacterium, preferably a Gram-positive bacterium.

An infection may be caused by Staphylococcus aureus. S. aureus is a Gram-positive, round-shaped bacterium belonging to the Firmicutes family, and is a usual member of the microbiota of the body, frequently found in the upper respiratory tract and on the skin. Although S. aureus can act as a commensal of the human microbiota, it is often pathogenic, being a common cause of infections as described elsewhere herein. Non-limiting examples of infection types caused by S. aureus include skin infections, lung infections (e.g. pneumonia), CNS infections (e.g. meningitis), bone infections (e.g. osteomyelitis), heart infections (e.g. endocarditis), toxic shock syndrome, joint infections (e.g. infectious arthritis), blood infections (e.g. bacteremia), sepsis, and the like. A Staphylococcus aureus may be a methicillin-resistant Staphylococcus aureus (MRSA). MRSA typically has a mecA gene producing an altered penicillin binding protein, PBP2a (also known as PBP2′). A Staphylococcus aureus is typically classified as MRSA when it displays an oxacillin MIC of 4 μg/mL or higher, and/or an cefoxitin MIC of 8 μg/mL or higher, which distinguishes MRSA from methicillin-sensitive Staphylococcus aureus (MSSA). MRSA is typically highly resistant to commonly used antibiotics, such as beta-lactams, co-trimoxazole, and aminoglycosides. MRSA is associated with healthcare-acquired (HA), community-acquired (CA), and livestock-acquired (LA) infections. Detection of MRSA may be performed by comparing its growth in the presence of antibiotics such as oxacillin or cefoxitin to the growth of MSSA, using culturing methods discussed in standard handbooks such as Schwalbe R. et al (supra), some of which are also demonstrated in the experimental section herein, and/or commercially available kits such as ETEST® (Biomerieux, NC, USA). Alternatively, MRSA may be detected using molecular phenotyping such as using PCR methods detecting mecA, as for example described in WO98/20157, WO99/16780, U.S. Pat. No. 5,702,895, WO02/82086 or multilocus sequence typing (MLST) as described in Enright et al., J Clin Microbiol 2000; 38:1008-15, all of which are incorporated by reference herein in their entireties. MLST involves sequencing of seven housekeeping genes (arcC, aroE, glpF, gmk, pta, tpi, yqiL) and assigning a sequence type (ST) number to isolates that are identical, allowing grouping of MRSA together in clonal complexes. Non-limiting examples of MRSA include the clonal complexes of S. aureus ST1-MRSA-IV, ST5-MRSA-I, ST5-MRSA-II, ST5-MRSA-IV, ST228-MRSA-I, ST8-MRSA-II, ST8-MRSA-IV, ST239-MRSA-III, ST247-MRSA-I, ST250-MRSA-I, ST22-MRSA-IV, ST36-MRSA-II, ST36-MRSA-IV, ST30-MRSA-IV, ST45-MRSA-IV, ST72-MRSA-IV, ST612-MRSA-IV, ST22-MRSA-IV, ST36-MRSA-II, ST36-MRSA-IV, and the like. Accordingly, in some embodiments the infection is caused by a methicillin-resistant Staphylococcus aureus.

A Staphylococcus aureus may be a vancomycin-resistant Staphylococcus aureus (VRSA) or vancomycin-intermediate Staphylococcus aureus (VISA). VRSA and VISA are typically resistant to commonly used antibiotics, especially glycopeptides such as vancomycin. A Staphylococcus aureus is typically classified as VISA when it displays a vancomycin MIC value of from 4 to 8 μg/mL, and VRSA when it displays a vancomycin MIC value of 16 μg/mL or higher, which distinguishes VISA and/or VRSA from vancomycin-sensitive Staphylococcus aureus (VSSA). A Staphylococcus aureus may be a heterogeneous vancomycin-intermediate Staphyloccocus aureus (hVISA), defined as Staphylococcus aureus that is resistant to vancomycin at a frequency of 10-6 colonies or higher. Detection of VISA and/or VRSA may be performed by comparing its growth in the presence of vancomycin to the growth of VSSA using culturing methods and/or commercially available kits as discussed elsewhere herein. Alternatively, VISA and/or VRSA may be detected using molecular phenotyping such as PCT methods detecting vanA, such as for example described in Dezfulian et al., Iran J Basic Med Sci. 2012; 15(2): 803-806 and Ramos-Trujillo et al., Int Microbiol 2003; 6(2):113-5, both of which are incorporated herein by reference in their entireties. Non-limiting examples of VISA and/or VRSA include S. aureus Mu3, Mu50, Mu50ω, SA137/93A, SA137/93G, JKD6001, JKD6005, JKD6008, JKD6023, JKD6051, PC-1, PC-3, MI, NJ, IL-A, IL-F, AMC11094, 99/3759-V, 99/3700-W, LIM-2, LIM-3, LIM-4, 28160, JH2, JH3, JH5, JH6, JH9, JH14, BR1, BR2, BR3, BR4, BR5, 98141, SF2, and the like. Accordingly, in some embodiments the infection is caused by vancomycin-intermediate and/or vancomycin-resistant Staphylococcus aureus.

A Staphylococcus aureus may be methicillin-resistant and vancomycin-intermediate and/or vancomycin-resistant. Accordingly, in some embodiments the infection is caused by methicillin-resistant and vancomycin-intermediate and/or vancomycin-resistant Staphylococcus aureus.

An infection may be a skin infection. The term “skin infection” as used herein has its customary and ordinary meaning as understood by one of skill in the art in view of this disclosure and refers to infection of the outer tissue covering the body of a vertebrate, preferably a mammal, more preferably a human, which may affect the associated soft tissues such as loose connective tissue and mucous membranes. The term skin infection as used herein also encompasses skin and skin structure infections (SSSIs), skin and soft tissue infections (SSTIs), and acute bacterial skin and skin structure infections SSSIs (ABSSSIs). Non-limiting examples of skin infections include cellulitis, erysipelas, folliculitis, furuncles, carbuncles, impetigo, erythrasma, MRSA skin infections, abscesses, ecthyma, staphylococcal scalded skin syndrome, pyomyositis, botryomycosis, necrotising fasciitis, gangrene, pitted keratolysis, chlamydia, gonorrhea, syphilis, vaginitis, candidiasis, napkin dermatitis, intertrigo, trichomycosis, paronychia, vulvovaginal candidiasis, malassezia folliculitis, aspergillosis, blastomycosis, cryptococcosis, chromoblastomycosis, histoplasmosis, mycetoma, sporotrichosis, systemic mycoses, talaromycosis, zyomycosis, mould infections, and the like.

In some embodiments, the skin infection is caused by a yeast or a filamentous fungus. In some embodiments, the skin infection is caused by a bacterium. Said bacterium may be Gram-positive or Gram-negative, preferably it is Gram-positive. A skin infection caused by a bacterium (bacterial skin infection) may be uncomplicated or complicated, mild or serious. Such infections may be without a lesion, abscess or wound (e.g., primary infections, such as all forms of impetigo including but not limited to mupirocin-resistant impetigo), or with a lesion, abscess or wound. Such infections may be of any size, including those with any lesion 75 cm2 or larger or lesser sized skin infections. Non-limiting examples of bacterial skin infections include Streptococcus (e.g. such as caused by Streptococcus pyogenes) infection, Staphylococcus (e.g. such as caused by Staphylococcus aureus) infection, methicillin-resistant Staphylococcus aureus (MRSA) infection, mupirocin-resistant MRSA infection, Enterococcus (e.g. such as caused by Enterococcus faecalis) infection, vancomycin-resistant bacteria infection, mupirocin-resistant bacteria infection, Clostridium (e.g. such as caused by Clostridium difficile) infection, Neisseria (e.g. such as caused by Neisseria gonorrhoeae) infection, Klebsiella (e.g. such as caused by Klebsiella pneumoniae) infection, drug-resistant malaria infection, multi-drug resistant (MDR) infection, extensively drug-resistant (XDR) tuberculosis infection, Escherichia coli (E. coli) infection, Corynebacterium infection, Brevibacterium infection, Shiga toxin-producing Escherichia coli (E. coli) infection, infections caused by bacteria possessing enzyme NDM-1 (New Delhi metallo-beta-lactamase-1), Mycobacterium (e.g. such as caused by Mycobacterium tuberculosis) infection, Mycoplasma (e.g. such as caused by Mycoplasma genitalium) infection, Campylobacter infection, gamma-proteobacteria infection, Enterobacteriaceae infection, carbapenem-resistant Enterobacteriaceae infection, Salmonella infection, Pseudomonas (e.g. such as caused by Pseudomonas aeruginosa) infection, Acinetobacter infection, and the like. In some embodiments, a skin infection is caused by Staphylococcus aureus. In some embodiments, a skin infection is caused by vancomycin-intermediate Staphylococcus aureus (VISA) and/or vancomycin-resistant Staphylococcus aureus (VRSA), as described elsewhere herein. In some embodiments, a skin infection is caused by methicillin-resistant Staphylococcus aureus (MRSA), as described elsewhere herein. In some embodiments, a skin infection is caused by methicillin-resistant and vancomycin-resistant and/or vancomycin-intermediate Staphylococcus aureus.

Within the context of the invention, one or more antimicrobial compounds such as antifungal agents, antiviral agents, essential oils, other bacteriocins, and/or antibiotics may be used in combination with a bacteriocin peptide or peptidomimetic. The skilled person understands that the choice of a particular antimicrobial compound will depend on the target organism. A bacteriocin may be any class I or class II bacteriocin, including any corresponding subgroup bacteriocin, as summarized in Cotter, P. D. et al., Nature Reviews Microbiology 2012; 11(2): 95-105, incorporated by reference herein in its entirety, and further discussed later herein. An antibiotic may be any compound selected from, but not limited to, the group of penicillins (β-lactams), aminonucleosides, nucleoside analogues, tetracyclines, cephalosporins, quinolones, lincomycins, macrolides, sulphonamides, polypeptides, glycopeptides, lipoglycopeptides, aminoglycosides, fluoroquinolones, monobactams, oxazolidinones, streptogramins, rifamycins, carbapenems, chloramphenicol, clindamycin, daptomycin, fosfomycin, lefamulin, metronidazole, mupirocin, nitrofurantoin, tigecycline, puromycin, hygromycin B (hygrovetine), geneticin (G418), bleomycin, zeocin, and blasticidin.

Within the context of the invention, the subject treated may be a vertebrate, preferably a mammal such as a cat, a mouse, a rat, a dog, or a human. In preferred embodiments, the subject treated is a human.

Within the context of the invention, administration of a bacteriocin peptide, peptidomimetic, or pharmaceutical composition as described herein may be performed to an individual, a cell, tissue, and/or an organ of an individual affected and/or at risk of developing an infection as discussed herein. Administration may be performed directly or indirectly in vivo, ex vivo or in vitro, using suitable means known in the art. When administering a bacteriocin peptide, peptidomimetic, or pharmaceutical composition as described herein, it is preferred that it is dissolved in a solution that is compatible with the delivery method. Such solutions are generally known in the art, see for example Remington: The Science and Practice of Pharmacy (supra). Improvements in means for providing an individual or a cell, tissue, and/or organ of said individual with the bacteriocin peptide, peptidomimetic and/or pharmaceutical composition are anticipated, considering the progress that has already thus far been achieved. Such future improvements may of course be incorporated to achieve the mentioned effect of the invention. The skilled person understands that the type and frequency of administration will vary depending on the infection. Administration may be one-time (single) or may involve multiple administrations over two, three, four, five, six, seven, eight, nine, ten days or more. Administration may be once daily or multiple times daily. Administration modes are generally known in the art. An administration mode may be topical, transdermal, intravenous, intramuscular, intraperitoneal, via inhalation, intraparenchymal, subcutaneous, intraarticular, intra-adipose tissue, oral, intrahepatic, intrasplanchnic, intra-ear, intrathoracic, intracardial, or intratracheal administration. A preferred administration mode for treatment, prevention and/or delaying of an infection, preferably a skin infection, as described herein is topical or transdermal administration. Topical administration suitably includes application to a tissue such as the skin surface. Transdermal application encompasses the fixation to the skin epithelium of transdermal patches that comprise a bacteriocin peptide, peptidomimetic, and/or pharmaceutical composition as described herein. Topical and/or transdermal administration may involve administration on a rash, wound, lesion, abscess, sore, blister, pimple, lump, wart, boil, and the like. Accordingly, in some embodiments, a bacteriocin peptide, peptidomimetic, or pharmaceutical composition is administered topically, preferably on a wound, lesion or abscess. In some embodiments, a bacteriocin peptide, peptidomimetic, or pharmaceutical composition is administered transdermally, preferably on a wound, lesion or abscess.

In some embodiments, a bacteriocin peptide, peptidomimetic, or pharmaceutical composition for use, methods, and uses according to the invention result in the alleviation of at least one symptom and/or the improvement of at least one parameter associated with an infection, preferably a skin infection. Alleviating a symptom of an infection, preferably a skin infection, as discussed herein may mean that said symptom is improved or decreased or that the progression of a typical symptom has been slowed down in an individual, in a cell, tissue or organ of said individual as assessed by a physician. A decrease or improvement of a typical symptom may mean a slowdown in progression of symptom development or a complete disappearance of symptoms. Symptoms, and thus also a decrease in symptoms, can be assessed using a variety of methods, to a large extent the same methods as used in diagnosis of the relevant infections, including clinical examination and routine laboratory tests. Laboratory tests may include both macroscopic and microscopic methods, molecular methods, radiographic methods such as X-rays, biochemical methods, immunohistochemical methods, culturing methods, and others. In this context, “decrease” (respectively “improvement”) means at least a detectable decrease (respectively a detectable improvement) using an assay known to a person of skill in the art. The decrease may be a decrease of at least 5%, at least 10%, at least 20%, at least 30%, at least 40%, at least 50%, at least 60%, at least 70%, at least 80%, at least 90% or at least 100%. The decrease may be seen after at least one day, at least two days, at least three days, at least four days, at least five days, at least six days, at least seven days, at least eight days, at least nine days, at least ten days or more of treatment using a bacteriocin peptide, peptidomimetic, and/or pharmaceutical composition as described herein. Typical symptoms associated with infection, preferably a skin infection, include fever, coughing, headache, nausea, vomiting, pain, the presence of a rash, lesion, sore, blister, pimple, lump, wart, abscess, or boil on the affected tissue, and the like. Improving a parameter may mean that the value of a typical parameter associated with an infection, preferably a skin infection, is improved in an individual, in a cell, tissue or organ of said individual as assessed by a physician. In this context, improvement of a parameter may be interpreted as to mean that said parameter assumes a value closer to the value displayed by a healthy individual. The improvement of a parameter may be seen after at least at least one day, at least two days, at least three days, at least four days, at least five days, at least six days, at least seven days, at least eight days, at least nine days, at least ten days or more of treatment using a bacteriocin peptide, peptidomimetic and/or pharmaceutical composition as described herein. Typical parameters associated with an infection, preferably a skin infection include fatigue, redness on the affected area, and the like.

Administration may involve a therapeutically effective amount of a peptide, peptidomimetic, or pharmaceutical composition as described herein. As used herein, an “effective amount” is an amount sufficient to exert beneficial or desired results. Accordingly, a “therapeutically effective amount” is an amount that, when administered to a subject such as a subject in need thereof, is sufficient to exert some therapeutic effect as described herein, such as, but not limited to, a reduction in the magnitude of at least one symptom and/or the improvement of at least one parameter associated with an infection, preferably a skin infection, as described earlier herein. An amount that is “therapeutically effective” will vary from subject to subject, depending on the age, the infection type and its progression, and overall general condition of the individual. An appropriate “therapeutically effective” amount in any individual case may be determined by the skilled person using routine experimentation, such as discussed elsewhere herein. A “subject in need” may be any individual affected by, and/or at risk of developing an infection, preferably a skin infection.

A bacteriocin peptide, peptidomimetic, or pharmaceutical composition may not inhibit or may partially inhibit, preferably it does not inhibit, a microbial cell, preferably a bacterium, normally present in a subject's microbiome, such as for example the skin microbiome. “Microbiome” or “flora” as used herein refers to the collective of microbial cells present in a healthy subject and/or tissue of a subject. Said microbial cells may be commensal or beneficial (mutualistic), i.e. exerting a health benefit to the subject. Non-limiting examples of such organisms are bacteria normally present in the gut such as probiotics (e.g. Lactobacillus, Bifidobacterium, Streptococcus, and the like) and bacteria normally present on the skin, such as, but not limited to, Staphylococcus epidermidis. In some embodiments, a preferred bacteriocin peptide, peptidomimetic, or pharmaceutical composition may not inhibit or may partially inhibit, preferably it does not inhibit, Staphylococcus epidermidis. A definition of “partial” inhibition, as well as methods for its determination, is provided earlier herein. As discussed elsewhere herein, the use of broad-spectrum antimicrobial compounds can also inhibit microbial cells normally present in the subject's microbiome, unbalancing the associated microbial communities and subsequently leaving their ecological niche(s) empty. When applied in treatment or cosmetic methods, this can promote the colonization of the empty niche(s) by other pathogenic organisms, or by the targeted pathogen after the acquisition of resistance to the antimicrobial compound. Staphylococcus epidermidis is a Gram-positive bacterium which is normally present in the human skin microbiome. The presence of Staphylococcus epidermidis on the skin is associated with several benefits. Besides inhibiting the growth of potential pathogens by natural competition, Staphylococcus epidermidis can inhibit Staphylococcus aureus growth by the production of serine protease glutamyl endopeptidase, and can induce keratinocytes to produce antimicrobial peptides via immune cell signalling, as discussed in Byrd et al., Nat Rev Microbiol 2018; 16:143-155, incorporated herein by reference in its entirety. Additionally, Staphylococcus epidermidis and other commensal skin bacteria such as Staphylococcus hominis can produce novel lantibiotics that are able to synergize with the human cathelicidin antimicrobial peptide LL-37 and can inhibit the growth of Staphylococcus aureus, as discussed in Nakatsuji et al., Sci. Transl Med. 2017; 9: eaah4680, incorporated herein by reference in its entirety. Lack of inhibition or partial inhibition of a microbial cell normally present in a subject's microbiome is, therefore, associated with at least one, or both, of the following benefits:
The normal microbiome of the subject is not inhibited, meaning that the associated microbial communities do not become imbalanced.
Beneficial microbial cells such as Staphylococcus epidermidis are not inhibited or are partially inhibited.

Accordingly, in some embodiments, a preferred bacteriocin peptide, peptidomimetic, or pharmaceutical composition does not inhibit or partially inhibits, preferably does not inhibit, at least one of the microbial cells, preferably at least one bacterium, normally present in the subject's microbiome. In preferred embodiments, a bacteriocin peptide, peptidomimetic, or pharmaceutical composition does not inhibit or partially inhibits, preferably does not inhibit, at least one of the microbial cells normally present on the subject's skin, preferably Staphylococcus epidermidis.

In some embodiments, a bacteriocin peptide, peptidomimetic, or pharmaceutical composition displays a ratio of inhibition of a target microbial cell, preferably a bacterium, more preferably Staphylococcus aureus, to inhibition of at least one of the microbial cells, preferably at least one bacterium, normally present in the subject's microbiome, more preferably Staphylococcus epidermidis, of 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 200 or more, 300 or more, 400 or more, 500 or more, 600 or more, 700 or more, 800 or more, 900 or more, 1000 or more, 10000 or more, 100000 or more, or 1000000 or more. Inhibition (i.e. biological activity) may be assessed as described elsewhere herein.

In some embodiments, in a bacteriocin peptide, peptidomimetic, pharmaceutical composition for uses, methods, uses as described herein at least 0.01 μg/ml, at least 0.1 μg/ml, at least 1 μg/ml, at least 2 μg/ml, at least 5 μg/ml, at least 10 μg/ml, at least 15 μg/ml, at least 16 μg/ml, at least 17 μg/ml, at least 18 μg/ml, at least 19 μg/ml, at least 20 μg/ml, at least 25 μg/ml, at least 30 μg/ml, at least 35 μg/ml, at least 40 μg/ml, at least 45 μg/ml, at least 50 μg/ml, at least 100 μg/ml, at least 200 μg/ml, preferably at least 15 μg/ml, of the peptide or peptidomimetic is present and/or used.

In a further aspect, the invention provides a bacteriocin peptide or peptidomimetic comprising, consisting essentially of, or consisting of, preferably comprising, a peptide or peptidomimetic represented by the amino acid sequence of any one of SEQ ID NOs: 7, 8, 15, 18, 19, 22, 24, 34, 37, 40, 42, 44, 47, 48, or 51.

In an embodiment, a bacteriocin peptide or peptidomimetic comprises, essentially consists of, or consists of, preferably comprises, a peptide or peptidomimetic represented by the amino acid sequence of SEQ ID NO: 7. In an embodiment, a bacteriocin peptide or peptidomimetic comprises, essentially consists of, or consists of, preferably comprises, a peptide or peptidomimetic represented by the amino acid sequence of SEQ ID NO: 8. In an embodiment, a bacteriocin peptide or peptidomimetic comprises, essentially consists of, or consists of, preferably comprises, a peptide or peptidomimetic represented by the amino acid sequence of SEQ ID NO: 15. In an embodiment, a bacteriocin peptide or peptidomimetic comprises, essentially consists of, or consists of, preferably comprises, a peptide or peptidomimetic represented by the amino acid sequence of SEQ ID NO: 18. In an embodiment, a bacteriocin peptide or peptidomimetic comprises, essentially consists of, or consists of, preferably comprises, a peptide or peptidomimetic represented by the amino acid sequence of SEQ ID NO: 19. In an embodiment, a bacteriocin peptide or peptidomimetic comprises, essentially consists of, or consists of, preferably comprises, a peptide or peptidomimetic represented by the amino acid sequence of SEQ ID NO: 22. In an embodiment, a bacteriocin peptide or peptidomimetic comprises, essentially consists of, or consists of, preferably comprises, a peptide or peptidomimetic represented by the amino acid sequence of SEQ ID NO: 24. In an embodiment, a bacteriocin peptide or peptidomimetic comprises, essentially consists of, or consists of, preferably comprises, a peptide or peptidomimetic represented by the amino acid sequence of SEQ ID NO: 34. In an embodiment, a bacteriocin peptide or peptidomimetic comprises, essentially consists of, or consists of, preferably comprises, a peptide or peptidomimetic represented by the amino acid sequence of SEQ ID NO: 37. In an embodiment, a bacteriocin peptide or peptidomimetic comprises, essentially consists of, or consists of, preferably comprises, a peptide or peptidomimetic represented by the amino acid sequence of SEQ ID NO: 40. In an embodiment, a bacteriocin peptide or peptidomimetic comprises, essentially consists of, or consists of, preferably comprises, a peptide or peptidomimetic represented by the amino acid sequence of SEQ ID NO: 42. In an embodiment, a bacteriocin peptide or peptidomimetic comprises, essentially consists of, or consists of, preferably comprises, a peptide or peptidomimetic represented by the amino acid sequence of SEQ ID NO: 44. In an embodiment, a bacteriocin peptide or peptidomimetic comprises, essentially consists of, or consists of, preferably comprises, a peptide or peptidomimetic represented by the amino acid sequence of SEQ ID NO: 47. In an embodiment, a bacteriocin peptide or peptidomimetic comprises, essentially consists of, or consists of, preferably comprises, a peptide or peptidomimetic represented by the amino acid sequence of SEQ ID NO: 48. In an embodiment, a bacteriocin peptide or peptidomimetic comprises, essentially consists of, or consists of, preferably comprises, a peptide or peptidomimetic represented by the amino acid sequence of SEQ ID NO: 51. A description of peptides and peptidomimetics according to the invention is given earlier herein.

### Compositions

A bacteriocin peptide or peptidomimetic as described herein exhibits a number of activities that can be advantageously used in a wide range of applications, including therapeutic applications, cosmetic applications, and applications in disinfection of surfaces (including surfaces of chemically fragile medical devices), biotechnology, biofermentation processes, and food preservation.

Provided herein, therefore, are compositions comprising a bacteriocin peptide or peptidomimetic as described earlier herein. Optionally, said compositions will further comprise an acceptable ingredient, such as a carrier, diluent, and/or excipient as discussed elsewhere herein. The skilled person is aware that each of the acceptable ingredients will be suitable for the intended use or application, for example pharmaceutical, cosmetic, or application in the disinfection of surfaces. In embodiments wherein the compositions comprise bacteriocin peptide or peptidomimetic salts as described earlier herein, the skilled person is aware that said salts will be suitable for the intended use or application, for example pharmaceutical, cosmetic, or application in the disinfection of surfaces. Non-limiting examples of pharmaceutically and cosmetically acceptable acids or bases suitable for the preparation of a bacteriocin or peptidomimetic salt as described earlier herein include organic and inorganic acids such as formic acid, acetic acid, propionic acid, lactic acid, glycolic acid, oxalic acid, pyruvic acid, succinic acid, maleic acid, malonic acid, trifluoroacetic acid, cinnamic acid, sulfuric acid, hydrochloric acid, hydrobromic acid, nitric acid, perchloric acid, phosphoric acid, and thiocyanic acid, which form ammonium salts with free amino groups of polypeptides, and bases that form carboxylate salts with free carboxylic groups of polypeptides, such as ethylamine, methylamine, dimethylamine, triethylamine, isopropylamine, diisopropylamine, and other mono-, di- and trialkylamines, and arylamines.

In an aspect, the invention provides a composition comprising a bacteriocin peptide or peptidomimetic as defined herein. In some embodiments, said composition is suitable for disinfecting a surface contaminated with Staphylococcus aureus, and optionally further comprises one or more antimicrobial compounds and/or a solvent. The skilled person understands that “suitable for disinfecting” in the context of the invention refers to said composition being capable of inhibiting Staphylococcus aureus, as defined earlier herein, when said composition is applied on a surface contaminated with said bacterium. A definition of a “contaminated surface” is provided later herein.

In some embodiments, the composition is a pharmaceutical composition comprising a bacteriocin peptide or peptidomimetic as defined herein, optionally further comprising one or more antimicrobial compounds and/or pharmaceutically acceptable ingredients.

In some embodiments, the composition is a cosmetic composition comprising a bacteriocin peptide or peptidomimetic as defined herein, optionally further comprising one or more antimicrobial compounds and/or cosmetically acceptable ingredients. Non-limiting examples of cosmetic compositions are skin care compositions, hair care compositions, face care compositions, personal care compositions, sun blockers, and the like. In some embodiments, a preferred cosmetic composition is a skin care composition.

Typically, compositions, pharmaceutical compositions, and cosmetic compositions as described herein comprise a bacteriocin peptide or peptidomimetic as described earlier herein at a concentration value from 0.01 to 1000 μg/ml, from 0.1 to 1000 g/ml, from 1 to 1000 μg/ml, from 2 to 500 μg/ml, from 3 to 125 μg/ml, from 4 to 75 μg/ml, or from 5 to 25 μg/ml, or from 10 to 20 μg/ml. Thus, a concentration value of a bacteriocin peptide or peptidomimetic may be at least 0.01 μg/ml, at least 0.1 μg/ml, at least 1 μg/ml, at least 2 μg/ml, at least 3 μg/ml, at least 4 μg/ml, at least 5 μg/ml, at least 6 μg/ml, at least 7 μg/ml, at least 8 μg/ml, at least 9 μg/ml, at least 10 μg/ml, at least 11 μg/ml, at least 12 μg/ml, at least 13 μg/ml, at least 14 μg/ml, at least 15 μg/ml, at least 16 μg/ml, at least 17 μg/ml, at least 18 μg/ml, at least 19 μg/ml, at least 20 μg/ml, at least 21 μg/ml, at least 22 μg/ml, at least 23 μg/ml, at least 24 μg/ml, at least 25 μg/ml, at least 26 μg/ml, at least 27 μg/ml, at least 28 μg/ml, at least 29 μg/ml, at least 30 μg/ml, at least 31 μg/ml, at least 32 μg/ml, at least 33 μg/ml, at least 34 μg/ml, at least 35 μg/ml, at least 36 μg/ml, at least 37 μg/ml, at least 38 μg/ml, at least 39 μg/ml, at least 40 μg/ml, at least 41 μg/ml, at least 42 μg/ml, at least 43 μg/ml, at least 44 μg/ml, at least 45 μg/ml, at least 46 μg/ml, at least 47 μg/ml, at least 48 μg/ml, at least 49 μg/ml, at least 50 μg/ml, at least 51 μg/ml, at least 52 μg/ml, at least 53 μg/ml, at least 54 μg/ml, at least 55 μg/ml, at least 56 μg/ml, at least 57 μg/ml, at least 58 μg/ml, at least 59 μg/ml, at least 60 μg/ml, at least 61 μg/ml, at least 62 μg/ml, at least 63 μg/ml, at least 64 μg/ml, at least 65 μg/ml, at least 66 μg/ml, at least 67 μg/ml, at least 68 μg/ml, at least 69 μg/ml, at least 70 μg/ml, at least 71 μg/ml, at least 72 μg/ml, at least 73 μg/ml, at least 74 μg/ml, at least 75 μg/ml, at least 76 μg/ml, at least 77 μg/ml, at least 78 μg/ml, at least 79 μg/ml, at least 80 μg/ml, at least 81 μg/ml, at least 82 μg/ml, at least 83 μg/ml, at least 84 μg/ml, at least 85 μg/ml, at least 86 μg/ml, at least 87 μg/ml, at least 88 μg/ml, at least 89 μg/ml, at least 90 μg/ml, at least 91 μg/ml, at least 92 μg/ml, at least 93 μg/ml, at least 94 μg/ml, at least 95 μg/ml, at least 96 μg/ml, at least 97 μg/ml, at least 98 μg/ml, at least 99 μg/ml, at least 100 μg/ml, at least 110 μg/ml, at least 120 μg/ml, at least 130 μg/ml, at least 140 μg/ml, at least 150 μg/ml, at least 160 μg/ml, at least 170 μg/ml, at least 180 g/ml, at least 190 μg/ml, at least 200 μg/ml, at least 250 μg/ml, at least 300 μg/ml, at least 400 μg/ml, at least 500 μg/ml, at least 550 μg/ml, at least 600 μg/ml, at least 650 μg/ml, at least 700 μg/ml, at least 750 μg/ml, at least 800 μg/ml, at least 850 μg/ml, at least 900 μg/ml, at least 950 μg/ml, or at least 1000 μg/ml, preferably it is at least 15 μg/ml.

Compositions, pharmaceutical compositions, and cosmetic compositions as described herein may be in any form as commonly used in the art. The skilled person is aware that the form of the respective composition will be suitable for the intended use or application, for example pharmaceutical, cosmetic, or application in the disinfection of surfaces. Non-limiting examples of suitable forms include tablets, capsules, pills, lyophilized, liquids, creams, ointments, gels, pastes, powders, emulsions, lotions, suspensions, sticks, aerosols (i.e. sprays), and the like.

Compositions, pharmaceutical compositions, and cosmetic compositions as described herein may optionally further comprise an antimicrobial compound. Said antimicrobial compound may be selected from any antimicrobial compound such as, but not limited to, antifungal agents, antiviral agents, essential oils, other bacteriocins, and/or antibiotics as described earlier herein. Non-limiting examples of suitable groups of bacteriocins are further given in Table 2.

TABLE 2
Examples of suitable groups of bacteriocins

| | | |
|---|---|---|
| Group | Distinctive feature | Examples |

Class I (typically modified)

| | | |
|---|---|---|
| MccC7-C51-type | Is covalently attached to a carboxy- | MccC7-C51 |
| bacteriocins | terminal aspartic acid | |
| Lasso peptides | Have a lasso structure | MccJ25 |
| Linear azole- or | Possess heterocycles but not other | MccB17 |
| azoline-containing | modifications | |
| peptides | | |
| Lantibiotics | Possess lanthionine bridges | Nisin, planosporicin, |
| | | mersacidin, actagardine, |
| | | mutacin 1140 |
| Linaridins | Have a linear structure and contain | Cypemycin |
| | dehydrated amino acids | |
| Proteusins | Contain multiple hydroxylations, | Polytheonamide A |
| | epimerizations and methylations | |
| Sactibiotics | Contain sulphur-α-carbon linkages | Subtilosin A, thuricin CD |
| Patellamide-like | Possess heterocycles and undergo | Patellamide A |
| cyanobactins | macrocyclization | |
| Anacyclamide-like | Cyclic peptides consisting of proteinogenic | Anacyclamide A10 |
| cyanobactins | amino acids with prenyl attachments | |
| Thiopeptides | Contain a central pyridine, dihydropyridine | Thiostrepton, nocathiacin I, |
| | or piperidine ring as well as heterocycles | GE2270 A, philipimycin |
| Bottromycins | Contain macrocyclic amidine, a | Bottromycin A2 |
| | decarboxylated carboxy-terminal thiazole | |
| | and carbon-methylated amino acids | |
| Glycocins | Contain S-linked glycopeptides | Sublancin 168 |

Class Il (typically unmodified or cyclic)

| | | |
|---|---|---|
| IIa peptides (pediocin | Possess a conserved YGNGV motif (in | Pediocin PA-1, enterocin |
| PA-1-like bacteriocins) | which N represents any amino acid) | CRL35, carnobacteriocin |
| | | BM1 |
| IIb peptides | Two unmodified peptides are required for | ABP118, lactacin F |
| | activity | |
| IIc peptides | Cyclic peptides | Enterocin AS-48 |
| IId peptides | Unmodified, linear, non-pediocin-like, | MccV, MccS, epidermicin |
| | single-peptide bacteriocins | NI01, lactococcin A |
| IIe peptides | Contain a serine-rich carboxy-terminal | MccE492, MccM |
| | region with a non-ribosomal siderophore- | |
| | type modification | |

As used herein, a “solvent” includes any solvent or mixture of solvents in which a bacteriocin peptide or peptidomimetic as described herein can be dissolved at a suitable concentration. The number and types of ionic charges in the peptide determine its solubility in aqueous solutions. In general, the more charged residues the peptide possesses, the more soluble it is in aqueous solutions. In addition, peptides generally have more charges at pH 6-8 than at pH 2-6. It is for this reason that peptides are generally better dissolved at near neutral pH. Among the many exceptions to the rule are peptide sequences that are very hydrophobic and those that tend to aggregate. While the hydrophobicity of the sequence is the primary cause of aggregation, peptides can also aggregate or “gel” through extensive hydrogen bonding network. Non-limiting examples of solvents that can be used in the context of the invention are water, ethanol, ammoniumhydroxide, dimethylsulfoxide (DMSO), acetic acid, acetonitrile and dimethylformamide (DMF). Dissolution can be enhanced by sonication.

A “pharmaceutical composition” is a composition which is suitable for use in therapy. A “cosmetic composition” is a composition which is suitable for use in personal care. As used herein, “pharmaceutically acceptable ingredients” and/or “cosmetically acceptable ingredients” include pharmaceutically and/or cosmetically acceptable carriers, fillers, preservatives, solubilizers, vehicles, diluents and/or excipients. Accordingly, the one or more pharmaceutically acceptable ingredients may be selected from the group consisting of pharmaceutically acceptable carriers, fillers, preservatives, solubilizers, vehicles, diluents and/or excipients. Such pharmaceutically acceptable carriers, fillers, preservatives, solubilizers, vehicles, diluents and/or excipients may be found in standard handbooks such as in Remington: The Science and Practice of Pharmacy (supra). Accordingly, the one or more cosmetically acceptable ingredients may be selected from the group consisting of cosmetically acceptable carriers, fillers, preservatives, solubilizers, vehicles, diluents and/or excipients. Such cosmetically acceptable carriers, fillers, preservatives, solubilizers, vehicles, diluents and/or excipients may be found in standard handbooks such as in Smolinske S. C., Handbook of Food, Drug, and Cosmetic Excipients, 1^{st} edition, CRC Press, USA (1992) and Sakamoto et al., Cosmetic Science and Technology: Theoretical Principles and Applications, 1^{st} edition, Elsevier, Netherlands (2017), both of which are incorporated herein by reference in their entireties.

A cosmetic composition according to the invention may be applied in various personal care and cleansing products, preferably skin care products, including but not limited to hand soap, hand hygiene, deodorants, face wash, body wash, shampoos, hair conditioner products, sun blockers, creams, lotions, ointments, gels, pastes, emulsions, suspensions, aerosols and the like.

Compositions, pharmaceutical compositions, and cosmetic compositions as described herein may optionally comprise additional compounds. Said compounds may help in delivery of the compositions. Suitable compounds in this context are: compounds capable of forming complexes, nanoparticles, micelles and/or liposomes that deliver each constituent as described herein, complexed or trapped in a vesicle or liposome through a cell membrane. Many of these compounds are known in the art. Suitable compounds comprise polyethylenimine (PEI), or similar cationic polymers, including polypropyleneimine or polyethylenimine copolymers (PECs) and derivatives; synthetic amphiphiles (SAINT-18); Lipofectin™, DOTAP. The skilled person will know which type of formulation is the most appropriate for a composition as described herein.

In some embodiments, compositions, pharmaceutical compositions, and/or cosmetic compositions as described herein do not comprise a bacteriocin peptide or peptidomimetic represented by the contiguous amino acid sequence GWGDVL (SEQ ID NO: 56) as the first 6 amino acids of its N-terminus end. In some embodiments, compositions, pharmaceutical compositions, and/or cosmetic compositions as described herein do not comprise a bacteriocin peptide or peptidomimetic represented by the contiguous amino acid sequence GWGDVL (SEQ ID NO: 56). In some embodiments, compositions, pharmaceutical compositions, and/or cosmetic compositions as described herein do not comprise a bacteriocin peptide or peptidomimetic comprising the contiguous amino acid sequence GWGDVL (SEQ ID NO: 56).

In some embodiments, in compositions, pharmaceutical compositions, and/or cosmetic compositions as described herein at least 0.01 μg/ml, at least 0.1 μg/ml, at least 1 μg/ml, at least 2 μg/ml, at least 5 g/ml, at least 10 μg/ml, at least 15 μg/ml, at least 16 μg/ml, at least 17 μg/ml, at least 18 μg/ml, at least 19 μg/ml, at least 20 μg/ml, at least 25 μg/ml, at least 30 μg/ml, at least 35 μg/ml, at least 40 μg/ml, at least 45 μg/ml, at least 50 μg/ml, at least 100 μg/ml, at least 200 μg/ml, preferably at least 15 μg/ml, of the bacteriocin peptide or peptidomimetic is present and/or used.

### Methods/Uses

In an aspect, the invention provides a cosmetic method of providing an improvement of skin hygiene comprising applying to the skin a bacteriocin peptide, peptidomimetic, or cosmetic composition, optionally further comprising one or more antimicrobial compounds and/or cosmetically acceptable ingredients, as defined herein.

In some embodiments, the bacteriocin peptide or peptidomimetic comprises, essentially consists of, or consists of, preferably comprises, a peptide or peptidomimetic represented by the amino acid sequence of SEQ ID NO: 2 or 3, preferably of SEQ ID NO: 2, or by an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with SEQ ID NO: 2 or 3, preferably of SEQ ID NO: 2.

In some embodiments, the bacteriocin peptide or peptidomimetic comprises, essentially consists of, or consists of, preferably comprises, a peptide or peptidomimetic represented by the amino acid sequence of any one of SEQ ID NOs: 7, 8, 15, 18, 19, 22, 24, 34, 37, 40, 42, 44, 47, 48, or 51, or by an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with any one of SEQ ID NOs: 7, 8, 15, 18, 19, 22, 24, 34, 37, 40, 42, 44, 47, 48, or 51.

A “cosmetic method” as used herein refers to a personal care method, i.e. a non-therapeutic method. “Applying to the skin” relates to the application of said peptide, peptidomimetic, or composition to the surface of the skin, in particular topical application or transdermal application, as described elsewhere herein. As a non-limiting example, when applied topically, a small quantity of a cosmetic composition as described herein, for example from 0.1 to 100 g, may be applied directly to the skin, optionally from a suitable container or applicator and, if necessary, may then be spread over and/or rubbed into the skin using the hand, fingers, or a suitable device. Accordingly, in preferred embodiments, the bacteriocin peptide, peptidomimetic, or cosmetic composition is applied topically or transdermally.

Application may be performed on the skin of a vertebrate, preferably a mammal such as a cat, a mouse, a rat, a dog, or a human. In preferred embodiments, application is performed on the skin of a human. Non-limiting examples of skin areas suitable for such application include the fingers, hands, toes, feet, face, nose, ears, forehead, torso, armpits, legs, arms, armpits, and the like. The skin may be wet or dry prior to application.

Application may be followed by a waiting period, wherein the bacteriocin peptide, peptidomimetic, or cosmetic composition is left in contact with the skin. Said period may last at least 15 seconds, at least 30 seconds, at least 45 seconds, at least 1 minute, at least 2 minutes, at least 5 minutes, at least 10 minutes, at least 25 minutes, at least 30 minutes, at least 1 hour, at least 2 hours, at least 3 hours, or more, preferably at least 15 seconds. After application or after the waiting period, the bacteriocin peptide, peptidomimetic, or cosmetic composition may be removed from the skin, e.g. by rinsing with water or by removing the transdermal patch if transdermal application is utilized, or be left on the skin.

Accordingly, in some embodiments, the invention provides a cosmetic method of providing an improvement of skin hygiene comprising applying to the skin a bacteriocin peptide, peptidomimetic, or cosmetic composition as defined herein, followed by a waiting period of at least 15 seconds.

The skilled person understands that “skin hygiene” in the context of the cosmetic method of the invention does not refer to a pathological condition, but to the maintenance of the skin's normal microbiome and overall cleanliness. The cosmetic method of the invention utilizes the antimicrobial effect of a bacteriocin peptide, peptidomimetic, or cosmetic composition as discussed elsewhere herein to improve the overall hygiene of healthy skin by removing undesirable microbial cells, akin to commercially available methods such as the application of soap to the skin. Removal of potentially pathogenic microbial cells, such as bacteria, from healthy skin is not necessarily prophylactic. Even in cases wherein potentially pathogenic microbial cells, such as a bacterium (for example Staphyloccocus aureus), are present on the skin, a healthy individual is not likely to develop a pathological state only because of the presence of such bacteria. For example, not disinfecting one's hands or armpits may result in undesirable microbial growth and effects such as unpleasant odor, but will not, as such lead to a pathological condition.

The cosmetic method is able to inhibit and/or kill a target microbial cell. The cosmetic method may result in at least a 2 log (a factor of 100), at least a 3 log (a factor of 1000), at least a 4 log (a factor of 10000), at least a 5 log (a factor of 100000), or at least a 6 log (a factor of 1000000) reduction of the non-inhibited and/or alive target microbial cell population, preferably bacterial population, more preferably Staphylococcus aureus population, on the skin.

The antimicrobial effect of a cosmetic method according to the invention may be assessed by standard methods in the art, such as commercial in vitro laboratory tests such as ASTM E2149-20 or ASTM E1054-08 (ASTM, PA, USA), and the like, or alternative methods discussed elsewhere herein. In some embodiments, the cosmetic method results in a reduction of the target microbial cell population, preferably bacterial population, more preferably Staphyloccocus aureus population, by at least 2-fold as assessed using the ASTM E1054-08 test in vitro.

A cosmetic method according to the invention may not inhibit or may partially inhibit, preferably it does not inhibit, a microbial cell, such as a bacterium like Staphylococcus epidermidis, normally present in a subject's skin microbiome. Definitions of “inhibition” and “microbiome” are provided earlier herein. As discussed elsewhere herein, lack of inhibition or partial inhibition of a microbial cell normally present in a subject's skin microbiome is associated with at least one, or both, of the following benefits:
The normal microbiome of the subject is not inhibited, meaning that the associated microbial communities do not become imbalanced.
Beneficial microbial cells such as Staphylococcus epidermidis are not inhibited or are partially inhibited.

Accordingly, in some embodiments, a preferred cosmetic method does not inhibit or partially inhibits, preferably does not inhibit, at least one of the microbial cells, preferably at least one bacterium, normally present in the subject's skin microbiome. In preferred embodiments, a cosmetic method does not inhibit or partially inhibits, preferably does not inhibit, Staphylococcus epidermidis.

In some embodiments, a cosmetic method displays a ratio of inhibition of a target microbial cell, preferably a bacterium, more preferably Staphylococcus aureus, to inhibition of at least one of the microbial cells, preferably at least one bacterium, normally present in the subject's skin microbiome, more preferably Staphylococcus epidermidis, of 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 200 or more, 300 or more, 400 or more, 500 or more, 600 or more, 700 or more, 800 or more, 900 or more, 1000 or more, 10000 or more, 100000 or more, or 1000000 or more. Inhibition (i.e. biological activity) may be assessed as described elsewhere herein.

In some embodiments, in cosmetic methods as described herein at least 0.01 μg/ml, at least 0.1 μg/ml, at least 1 μg/ml, at least 2 μg/ml, at least 5 μg/ml, at least 10 μg/ml, at least 15 μg/ml, at least 16 μg/ml, at least 17 μg/ml, at least 18 μg/ml, at least 19 μg/ml, at least 20 μg/ml, at least 25 g/ml, at least 30 μg/ml, at least 35 μg/ml, at least 40 μg/ml, at least 45 μg/ml, at least 50 μg/ml, at least 100 μg/ml, at least 200 μg/ml, preferably at least 15 μg/ml, of the bacteriocin peptide or peptidomimetic is present and/or used.

In an aspect, the invention provides an ex-vivo method of disinfecting a surface comprising contacting said surface with a bacteriocin peptide, peptidomimetic, or a composition, wherein said composition is suitable for disinfecting a surface contaminated with Staphylococcus aureus, optionally further comprising one or more antimicrobial compounds and/or a solvent as defined herein. In some embodiments, a preferred surface is contaminated with a bacterium, preferably with Staphylococcus aureus.

In some embodiments, the bacteriocin peptide or peptidomimetic comprises, essentially consists of, or consists of, preferably comprises, a peptide or peptidomimetic represented by the amino acid sequence of SEQ ID NO: 2 or 3, preferably of SEQ ID NO: 2, or by an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with SEQ ID NO: 2 or 3, preferably of SEQ ID NO: 2.

In some embodiments, the bacteriocin peptide or peptidomimetic comprises, essentially consists of, or consists of, preferably comprises, a peptide or peptidomimetic represented by the amino acid sequence of any one of SEQ ID NOs: 7, 8, 15, 18, 19, 22, 24, 34, 37, 40, 42, 44, 47, 48, or 51, or by an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with any one of SEQ ID NOs: 7, 8, 15, 18, 19, 22, 24, 34, 37, 40, 42, 44, 47, 48, or 51.

A “surface” as used herein refers to any non-living, preferably solid, surface which may serve as scaffold (i.e. provide physical support) for microbial growth. The method is further applicable both to the disinfection of instruments, such as medical instruments, and articles placed in small disinfection chambers, biological safety cabinets, isolators, glove boxes, incubators, materials airlocks, and the like. The method is also applicable for disinfection of food containers, industrial equipment and the like. In some embodiments, the surface is a surface of a medical instrument. Non-limiting examples of medical instruments include bedpans, cannulas, cardioverters, defibrillators, catheters, dialysers, electrocardiogramachines, enema equipment, endoscopes, gas cylinders, gauze sponges, surgical scissors, hypodermic needles, syringes, infection control equipment such as masks, gowns, face shields, and goggles, instrument sterilizers, kidney dishes, nasogastric tubes, nebulizers, ophthalmoscopes, otoscopes, pipettes, proctoscopes, radiographers, sphygmomanometers, thermometers, tongue depressors, transfusion kits, tuning forks, ventilators, watches, and the like. In some embodiments, the surface is the surface of industrial equipment. Non-limiting examples of industrial equipment include fermentation equipment, such as fermenters, tubing, feeding vessels, spargers, mixers, compressors, and the like, freezers, fridges, cargo vehicles, storage vessels, rotor blades, mills, and the like. A surface is “contaminated” with a microbial cell, such as Staphylococcus aureus or another bacterium, when its presence can be detected on said surface, using standard methods in the art such as swab tests.

“Disinfection”, otherwise known as “decontamination” has its customary and ordinary meaning as understood by one of skill in the art in view of this disclosure. It generally refers to the inhibition and/or killing of microbial cells on inert surfaces. Disinfection may be partial, i.e. a part of the target microbial cell population may not be neutralized and/or killed. Partial disinfection may mean that at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 99% of the targeted population is not inhibited and/or killed.

The ex-vivo method may result in at least a 2 log (a factor of 100), at least a 3 log (a factor of 1000), at least a 4 log (a factor of 10000), at least a 5 log (a factor of 100000), or at least a 6 log (a factor of 1000000) reduction of the non-inhibited and/or alive target microbial cell population, preferably bacterial population, more preferably Staphylococcus aureus population, on the surface.

Contacting with a surface may be followed by a waiting period, wherein the bacteriocin peptide, peptidomimetic, or composition is left in contact with the surface. Said period may last at least 15 seconds, at least 30 seconds, at least 45 seconds, at least 1 minute, at least 2 minutes, at least 5 minutes, at least 10 minutes, at least 25 minutes, at least 30 minutes, at least 1 hour, at least 2 hours, at least 3 hours, at least 4 hours, at least 5 hours, at least 6 hours, at least 12 hours, at least 24 hours, or more. After contacting with a surface or after the waiting period, the bacteriocin peptide, peptidomimetic or composition may be removed from the surface, e.g. by rinsing with water or by wiping said surface with a clean cloth.

Accordingly, in some embodiments, the invention provides an ex-vivo method of disinfecting a surface, preferably a surface contaminated with a bacterium, more preferably a surface contaminated with Staphyloccocus aureus, comprising contacting said surface with a bacteriocin peptide, peptidomimetic, or a composition, wherein said composition is suitable for disinfecting a surface contaminated with Staphylococcus aureus, optionally further comprising one or more antimicrobial compounds and/or a solvent as defined herein, followed by a waiting period of at least 15 seconds.

The antimicrobial effect of an ex-vivo method according to the invention may be assessed by standard methods in the art, such as commercial in vitro laboratory tests such as ASTM E2149-20 or ASTM E1054-08 (ASTM, PA, USA), and the like, or alternative methods discussed elsewhere herein. In some embodiments, the ex-vivo method results in a reduction of the target microbial cell population, preferably bacterial population, more preferably Staphylococcus aureus population, by at least 2-fold as assessed using the ASTM E1054-08 test in vitro.

In some embodiments, in ex-vivo methods as described herein at least 0.01 μg/ml, at least 0.1 μg/ml, at least 1 μg/ml, at least 2 μg/ml, at least 5 μg/ml, at least 10 μg/ml, at least 15 g/ml, at least 16 μg/ml, at least 17 μg/ml, at least 18 μg/ml, at least 19 μg/ml, at least 20 μg/ml, at least 25 μg/ml, at least 30 μg/ml, at least 35 μg/ml, at least 40 μg/ml, at least 45 μg/ml, at least 50 μg/ml, at least 100 μg/ml, at least 200 μg/ml, preferably at least 15 μg/ml, of the bacteriocin peptide or peptidomimetic is present and/or used.

Within the context of a bacteriocin peptide, peptidomimetic, pharmaceutical composition for use, uses, methods, cosmetic methods, and ex-vivo methods described herein, the target microbial cell population, preferably bacterial population, more preferably Staphylococcus aureus population, may be comprised in a biofilm. A “biofilm” comprises any syntrophic consortium of microbial cells, preferably bacteria, more preferably Staphylococcus aureus, in which cells stick to each other and often also to a surface. Typically, biofilms are resistant to commonly used antimicrobials, such as antibiotics.

### General Information

Unless stated otherwise, all technical and scientific terms used herein have the same meaning as customarily and ordinarily understood by a person of ordinary skill in the art to which this invention belongs, and read in view of this disclosure.

### Sequence Identity

In the context of the invention, a nucleic acid encoding a bacteriocin peptide is represented by a nucleotide sequence. In the context of the invention, a bacteriocin peptide or peptidomimetic is represented by an amino acid sequence. It is to be understood that each nucleic acid molecule or peptide or peptidomimetic as identified herein by a given sequence identity number (SEQ ID NO) is not limited to said specific sequence as disclosed.

Throughout this application, each time one refers to a specific nucleotide sequence SEQ ID NO (take SEQ ID NO: X as example) encoding a given protein fragment or polypeptide or peptide or derived peptide, one may replace it by:
i. a nucleotide sequence comprising a nucleotide sequence that has at least 60%, 70%, 80%, 90%, 95% or 99% sequence identity with SEQ ID NO: X;
ii. a nucleotide sequence the sequence of which differs from the sequence of a nucleic acid molecule of (i) due to the degeneracy of the genetic code; or
iii. a nucleotide sequence that encodes an amino acid sequence that has at least 60%, 70%, 80%, 90%, 95% or 99% amino acid identity or similarity with an amino acid sequence encoded by a nucleotide sequence SEQ ID NO: X.

Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Throughout this application, each time one refers to a specific amino acid sequence SEQ ID NO (take SEQ ID NO: Y as example), one may replace it by: a polypeptide represented by an amino acid sequence comprising a sequence that has at least 60%, 70%, 80%, 90%, 95% or 99% sequence identity or similarity with amino acid sequence SEQ ID NO: Y. Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Each nucleotide sequence or amino acid sequence described herein by virtue of its identity or similarity percentage with a given nucleotide sequence or amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively.

The terms “homology”, “sequence identity” and the like are used interchangeably herein. Sequence identity is described herein as a relationship between two or more amino acid (peptide, polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004, each incorporated by reference herein in its entirety.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity (as described below).

A global alignment is suitably used to determine sequence similarity or identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919, incorporated herein by reference in its entirety).

Alternatively percentage similarity or identity may be determined by searching against public databases, using algorithms such as FASTA, BLAST, etc. Thus, the nucleic acid and protein sequences of some embodiments of the present invention can further be used as a “query sequence” to perform a search against public databases to, for example, identify other family members or related sequences. Such searches can be performed using the BLASTn and BLASTx programs (version 2.0) of Altschul, et al., J. Mol. Biol. 215:403-10 (1990), incorporated herein by reference in its entirety. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., Nucleic Acids Res. 25(17): 3389-3402 (1997), incorporated herein by reference in its entirety. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.gov/. The above algorithms may also be used to determine corresponding nucleotide or amino acid residue positions between sequences being aligned. For example, an amino acid residue in sequence Y which corresponds to position 1 (or any other position) of sequence X may be determined.

Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

### Alternative Conservative Amino Acid Residue Substitution Classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, |
| | and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, P |
| formation | and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

### Codon Optimization

“Codon optimization”, as used herein, refers to the processes employed to modify an existing coding sequence, or to design a coding sequence, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host cell. For example, to suit the codon preference of mammalian, insect, plant, or microbial cells, preferably microbial cells. Examples of microbial cells include eukaryotes such as yeasts, filamentous fungi, and algae, and prokaryotes such as bacteria and archaea. Codon optimization also eliminates elements that potentially impact negatively RNA stability and/or translation (e. g. termination sequences, TATA boxes, splice sites, ribosomal entry sites, repetitive and/or GC rich sequences and RNA secondary structures or instability motifs).

### Proteins and Amino Acids

The terms “protein” or “peptide” or “amino acid sequence” are used interchangeably and refer to molecules consisting of a chain of amino acids, without reference to a specific mode of action, size, 3-dimensional structure or origin. In amino acid sequences as described herein, amino acids or “residues” are denoted by three-letter symbols. These three-letter symbols as well as the corresponding one-letter symbols are well known to a person of skill in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, Y (Tyr) is tyrosine. A residue may be any proteinogenic amino acid, but also any non-proteinogenic amino acid such as D-amino acids and modified amino acids formed by post-translational modifications, and also any non-natural amino acid.

### Peptide Expression

Peptide “expression” or “production” by a cell may be assessed by any method known to a person of skill in the art. For example, expression may be assessed by measuring the levels of gene expression on the level of the mRNA or the peptide by standard assays known to a person of skill in the art, such as qPCR, RNA sequencing, Northern blot analysis, Western blot analysis, mass spectrometry analysis of protein-derived peptides or ELISA.

### General Terms

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” or “to essentially consist of”. meaning that a composition as described herein may comprise additional component(s) than the ones specifically identified, said additional component(s) not altering the unique characteristics of the invention. In addition, the verb “to consist” may be replaced by “to consist essentially of” meaning that a method or use as described herein may comprise additional step(s) than the ones specifically identified, said additional step(s) not altering the unique characteristic of the invention. In addition, the verb “to consist” may be replaced by “to consist essentially of” meaning that a nucleotide or amino acid sequence as described herein may comprise additional nucleotides or amino acids than the ones specifically identified, said additional nucleotides or amino acids not altering the unique characteristics of the invention.

Reference to an element by the indefinite article “a” or “an” does not exclude the possibility that more than one of the element is present, unless the context clearly requires that there be one and only one of the elements. The indefinite article “a” or “an” thus usually means “at least one”. As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 1% of the value. As used herein, the term “and/or” indicates that one or more of the stated cases may occur, alone or in combination with at least one of the stated cases, up to with all of the stated cases.

Various embodiments are described herein. Each embodiment as identified herein may be combined together unless otherwise indicated.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

One skilled in the art will recognize many methods and materials similar or equivalent to those described herein, which could be used in the practice of the present invention. Indeed, the present invention is in no way limited to the methods and materials described.

The present invention is further described by the following examples which should not be construed as limiting the scope of the invention.

### DESCRIPTION OF THE FIGURES

FIG. 1. Biological activity of a bacteriocin peptide represented by SEQ ID NO: 2 (cerein 7B) against Staphylococcus aureus ATCC 6538. 2 μl of an aqueous solution of said peptide (1 mg/ml concentration) was deposited on an inoculated M17 agar plate. After overnight incubation, an inhibition halo was visible on the spot of the deposit.

FIG. 2. Biological activity of a bacteriocin peptide represented by SEQ ID NO: 2 (cerein 7B) against Staphylococcus epidermidis ATCC 12228. 2 μl of an aqueous solution of said peptide (1 mg/ml concentration) was deposited on an inoculated M17 agar plate. An equivalent amount of aureocin A53 aqueous solution (1 mg/ml concentration) was used as positive control. After overnight incubation, no inhibition halo was visible on the spot of the deposit of the aqueous solution of cerein 7B, whereas an inhibition halo was present on the spot of the aureocin A53 deposit.

FIG. 3. MIC assay of a bacteriocin peptide represented by SEQ ID NO: 2 (cerein 7B) against Staphylococcus aureus ATCC 6538. OD₆₀₀ values of the different cultures without the addition of bacteriocin (black line), were compared to cultures that were exposed to varying concentration of bacteriocin (dotted line). In well number 7 it is shown that a concentration of 15.63 μg/ml was able to inhibit 50% of culture growth. Bacteriocin concentration was 250 μg/ml in well number 11, diluted 2-fold per subsequent well (see also Table 3).

FIG. 4. MIC assay of a bacteriocin peptide represented by SEQ ID NO: 2 (cerein 7B) against Staphylococcus epidermidis ATCC 12228. OD₆₀₀ values of the different cultures without the addition of bacteriocin (black line), were compared to cultures that were exposed to varying concentration of bacteriocin (dotted line). No growth inhibition was observed with any of the tested concentrations. Bacteriocin concentration was 250 μg/ml in well number 11, diluted 2-fold per subsequent well (see also Table 3).

FIG. 5. Generation of a bacteriocin mutant library by alanine scanning and assessment of their antimicrobial activity. Mutant peptides were generated by single substitutions of amino acids of SEQ ID NO: 2 by alanine residues. Wild type (A1) denotes cerein 7B (SEQ ID NO: 2). Corresponding SEQ ID NO for each mutant is given in Table 4. Black cells denote the presence and white cells denote the absence of antimicrobial activity against S. aureus ATCC 6538 in M17 agar plates after overnight incubation.

FIG. 6. Activity test of mutants against S. aureus ATCC 6538. 5 μl of peptide (SEQ ID NO: 2, 4-51) aqueous solutions at a concentration of 1 mg/ml were deposited on M17 agar plates of S. aureus ATCC 6538. Cerein 7B (SEQ ID NO: 2) was tested in position A1 (white circle). Tested positions for peptides corresponding to SEQ ID NO: 4-51 (mutants 1-48) correspond to the names shown in brackets in Table 4. Corresponding SEQ ID NO for each mutant is given in Table 4. After overnight incubation, the presence of an inhibition halo was considered as a positive result of antimicrobial activity.

### EXAMPLES

### General Procedures to the Examples

### Bacteriocin Peptide Synthesis

For biological activity tests on agar plates performed in Example 1 and MIC assays performed in Example 2, a bacteriocin peptide (cerein 7B, SEQ ID NO: 2) was chemically synthesized (Peptide 2.0, VA, USA).

For mutant peptide synthesis used in the experiments performed in Example 3, bacteriocin (SEQ ID NO: 2, 4-51) peptides were synthesized in vitro (Gabant et al., 2019). Amino acid sequences were reverse-translated and codon optimized for Escherichia coli expression. (www.bioinformatics.org/sms2/rev_trans.html). The nucleotide sequences were cloned in a pUC57 vector backbone (Thermo-Scientific, MA, USA), comprising the T7 promoter, a start codon (ATG) and stop codon (TAA), and the T7 terminator region. Recombinant vectors were cloned in E. coli DH10B standard strain and used as templates for cell-free protein synthesis using PURExpress® in vitro Protein Synthesis Kit (New England Biolabs, MA, USA) following the manufacturer's protocol.

### Culture Growth Conditions

Bacterial strains were grown overnight in liquid culture on rotary shakers or on agar plates using M17 media supplemented with 0.5% glucose. S. aureus ATCC 6538 and S. epidermidis ATCC 12228 liquid cultures and agar plates were grown anaerobically at 37° C. Culture volume utilized in MIC assays was 200 μl.

### MIC Assays

Determination of MIC values of a cerein 7B peptide (SEQ ID NO: 2) was performed in liquid cultures of S. aureus ATCC 6538 and S. epidermidis ATCC 12228. Growth was assessed by measuring endpoint OD₆₀₀ values in a Spectramax i3 (Molecular Devices, CA, USA). Minimum inhibitory concentration (MIC) was defined as the lowest concentration which inhibited 50% of the growth of cultures as compared to reference cultures not exposed to the peptide. Concentration of the peptide was 250 μg/ml in well 11, diluted 2-fold per subsequent well as shown in Table 3 (concentrations rounded to the second decimal):

TABLE 3
Cerein 7B peptide concentration (SEQ ID
NO: 2) concentration value per well

| Well number | Concentration (μg/ml) |
|---|---|
| 11 | 250 |
| 10 | 125 |
| 9 | 62.5 |
| 8 | 31.25 |
| 7 | 15.63 |
| 6 | 7.81 |
| 5 | 3.91 |
| 4 | 1.95 |
| 3 | 0.98 |
| 2 | 0.49 |
| 1 | 0.24 |

### Biological Activity Tests

Antimicrobial activity of bacteriocin peptides was tested on M17 agar plates in Examples 1 and 3.

In Example 1, 2 μl of cerein 7B peptide (SEQ ID NO: 2) aqueous solutions at a concentration of 1 mg/ml were deposited on inoculated M17 agar plates of S. aureus ATCC 6538 and S. epidermidis ATCC 12228. In the case of S. epidermidis ATCC 12228, an equivalent amount of aureocin A53 aqueous solution (1 mg/ml concentration) was used as positive control.

In Example 3, 5 μl of bacteriocin peptides (SEQ ID NO: 2, 4-51) aqueous solutions at a concentration of 1 mg/ml were deposited on inoculated M17 agar plates of S. aureus ATCC 6538. After overnight incubation, the presence of an inhibition halo was considered as a positive result of antimicrobial activity.

### Example 1. Biological Activity

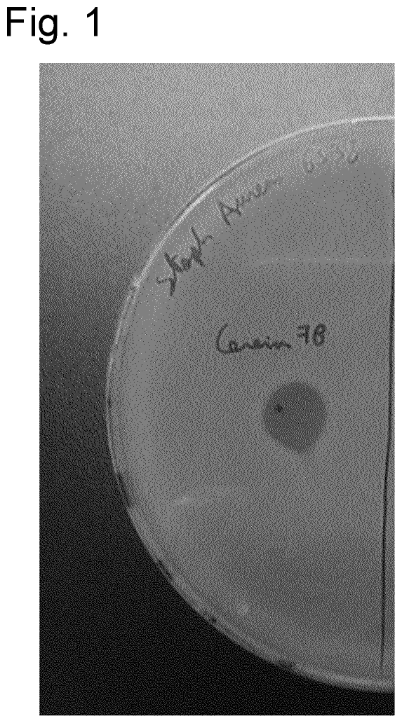

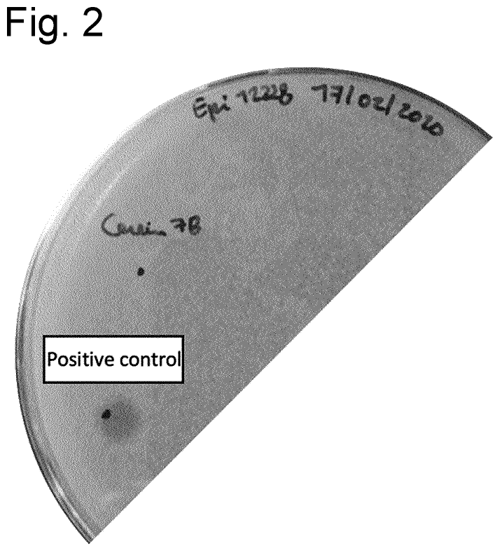

Antimicrobial activity of a cerein 7B peptide (SEQ ID NO: 2) was initially assessed on inoculated M17 agar plates of S. aureus ATCC 6538 and S. epidermidis ATCC 12228. An inhibition halo was visible in the case of S. aureus ATCC 6538 whereas no inhibition halo was present in the case of S. epidermidis ATCC 12228. These results (FIGS. 1 and 2) confirm that cerein 7B is able to inhibit S. aureus ATCC 6538 without inhibiting S. epidermidis ATCC 12228.

### Example 2. MIC Value Determination Against S. aureus

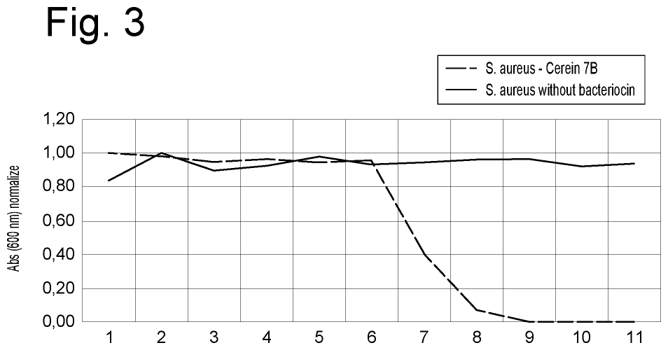

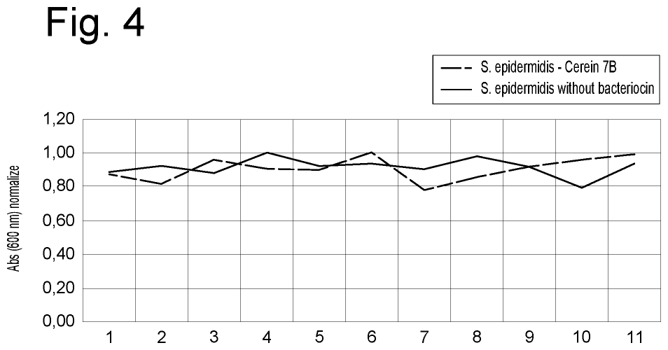

To determine the MIC value of cerein 7B (SEQ ID NO: 2) against S. aureus, the effect of addition of varying concentrations of cerein 7B was evaluated in liquid cultures. As shown in FIG. 3, cerein 7B is able to inhibit S. aureus ATCC 6538 at a MIC value of 15.63 μg/ml. In accordance with the biological activity determination experimental results, no inhibition of S. epidermidis ATCC 12228 was observed at any of the cerein 7B concentrations tested (FIG. 4).

### Example 3. Determination of Biological Activity of a Bacteriocin Mutant Library

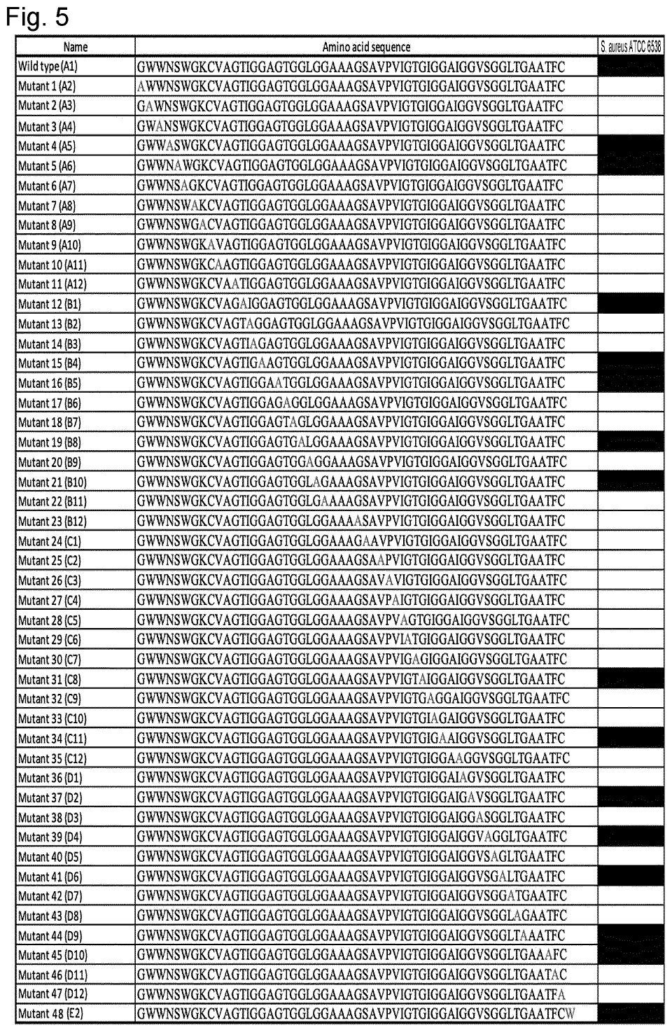

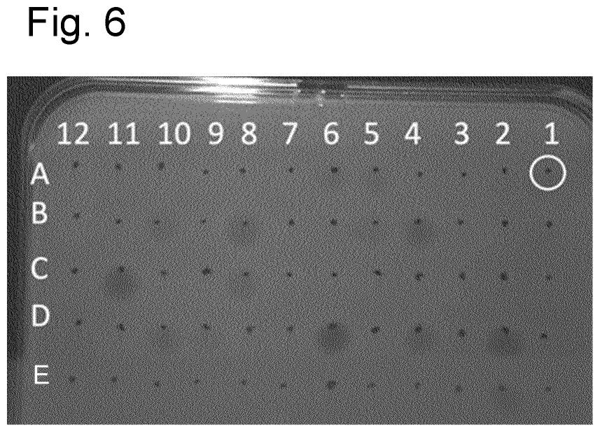

To determine the contribution of specific amino acid residues of cerein 7B (SEQ ID NO: 2) to its activity against microbial target cells, a total of 47 bacteriocin peptide mutants (SEQ ID NO: 4-50) were generated by single substitutions of amino acids by alanine residues. An additional length mutant was generated (SEQ ID NO: 51) by addition of a tryptophan residue at the C-terminus of SEQ ID NO: 2. The biological activity of all mutants was assessed on inoculated M17 agar plates of S. aureus ATCC 6538 (FIGS. 5 and 6). The inventors determined that a significant number of amino acid residues of SEQ ID NO: 2 can be substituted by an alanine without compromising antimicrobial activity (FIGS. 5 and 6). Further, addition of a tryptophan residue at the C-terminus of SEQ ID NO: 2 did not compromise its antimicrobial activity (FIGS. 5 and 6). Specifically, the results demonstrate that at least the non-underlined amino acid residues of SEQ ID NO: 2 as demonstrated below may be substituted without compromising of the antimicrobial activity of cerein 7B:

GWWNSWGKCVAGTIGGAGTGGLGGAAAGSAVPVIGTGIGGAIGGVSGGL
TGAATFC

Overall, the present results demonstrate the strong potential of bacteriocin peptides and peptidomimetics as described herein as antimicrobial compounds that can selectively inhibit their microbial cell targets.

TABLE 4
Sequences

| | | Amino | |
|---|---|---|---|
| SEQ ID | | acid (aa) | |
| NO: | Name | or DNA | Sequence |
| 1 | cerein 7B with | aa | MENLQMLTEEELMEIEGGGWWNSWGKCVAGTIGGAGTG |
| | signal pept | | GLGGAAAGSAVPVIGTGIGGAIGGVSGGLTGAATFC |
| 2 | cerein 7B | aa | GWWNSWGKCVAGTIGGAGTGGLGGAAAGSAVPVIGTGIG |
| | | | GAIGGVSGGLTGAATFC |
| 3 | bacteriocin | aa | GWWXaaXaaWGKCVXaaGXaaIGXaaXaaXaaTGXaaLXaaG |
| | consensus | | XaaXaaXaaGSXaaVPVIGTXaaIGXaaXaaIGXaaVXaaGXaaL |
| | | | TXaaXaaXaaXaaFC |
| 4 | mutant 1 (A2) | aa | AWWNSWGKCVAGTIGGAGTGGLGGAAAGSAVPVIGTGIG |
| | | | GAIGGVSGGLTGAATFC |
| 5 | mutant 2 (A3) | aa | GAWNSWGKCVAGTIGGAGTGGLGGAAAGSAVPVIGTGIG |
| | | | GAIGGVSGGLTGAATFC |
| 6 | mutant 3 (A4) | aa | GWANSWGKCVAGTIGGAGTGGLGGAAAGSAVPVIGTGIG |
| | | | GAIGGVSGGLTGAATFC |
| 7 | mutant 4 (A5) | aa | GWWASWGKCVAGTIGGAGTGGLGGAAAGSAVPVIGTGIG |
| | | | GAIGGVSGGLTGAATFC |
| 8 | mutant 5 (A6) | aa | GWWNAWGKCVAGTIGGAGTGGLGGAAAGSAVPVIGTGIG |
| | | | GAIGGVSGGLTGAATFC |
| 9 | mutant 6 (A7) | aa | GWWNSAGKCVAGTIGGAGTGGLGGAAAGSAVPVIGTGIG |
| | | | GAIGGVSGGLTGAATFC |
| 10 | mutant 7 (A8) | aa | GWWNSWAKCVAGTIGGAGTGGLGGAAAGSAVPVIGTGIG |
| | | | GAIGGVSGGLTGAATFC |
| 11 | mutant 8 (A9) | aa | GWWNSWGACVAGTIGGAGTGGLGGAAAGSAVPVIGTGIG |
| | | | GAIGGVSGGLTGAATFC |
| 12 | mutant 9 (A10) | aa | GWWNSWGKAVAGTIGGAGTGGLGGAAAGSAVPVIGTGIG |
| | | | GAIGGVSGGLTGAATFC |
| 13 | mutant 10 | aa | GWWNSWGKCAAGTIGGAGTGGLGGAAAGSAVPVIGTGIG |
| | (A11) | | GAIGGVSGGLTGAATFC |
| 14 | mutant 11 | aa | GWWNSWGKCVAATIGGAGTGGLGGAAAGSAVPVIGTGIG |
| | (A12) | | GAIGGVSGGLTGAATFC |
| 15 | mutant 12 (B1) | aa | GWWNSWGKCVAGAIGGAGTGGLGGAAAGSAVPVIGTGIG |
| | | | GAIGGVSGGLTGAATFC |
| 16 | mutant 13 (B2) | aa | GWWNSWGKCVAGTAGGAGTGGLGGAAAGSAVPVIGTGI |
| | | | GGAIGGVSGGLTGAATFC |
| 17 | mutant 14 (B3) | aa | GWWNSWGKCVAGTIAGAGTGGLGGAAAGSAVPVIGTGIG |
| | | | GAIGGVSGGLTGAATFC |
| 18 | mutant 15 (B4) | aa | GWWNSWGKCVAGTIGAAGTGGLGGAAAGSAVPVIGTGIG |
| | | | GAIGGVSGGLTGAATFC |
| 19 | mutant 16 (B5) | aa | GWWNSWGKCVAGTIGGAATGGLGGAAAGSAVPVIGTGIG |
| | | | GAIGGVSGGLTGAATFC |
| 20 | mutant 17 (B6) | aa | GWWNSWGKCVAGTIGGAGAGGLGGAAAGSAVPVIGTGIG |
| | | | GAIGGVSGGLTGAATFC |
| 21 | mutant 18 (B7) | aa | GWWNSWGKCVAGTIGGAGTAGLGGAAAGSAVPVIGTGIG |
| | | | GAIGGVSGGLTGAATFC |
| 22 | mutant 19 (B8) | aa | GWWNSWGKCVAGTIGGAGTGALGGAAAGSAVPVIGTGIG |
| | | | GAIGGVSGGLTGAATFC |
| 23 | mutant 20 (B9) | aa | GWWNSWGKCVAGTIGGAGTGGAGGAAAGSAVPVIGTGI |
| | | | GGAIGGVSGGLTGAATFC |
| 24 | mutant 21 | aa | GWWNSWGKCVAGTIGGAGTGGLAGAAAGSAVPVIGTGIG |
| | (B10) | | GAIGGVSGGLTGAATFC |
| 25 | mutant 22 | aa | GWWNSWGKCVAGTIGGAGTGGLGAAAAGSAVPVIGTGIG |
| | (B11) | | GAIGGVSGGLTGAATFC |
| 26 | mutant 23 | aa | GWWNSWGKCVAGTIGGAGTGGLGGAAAASAVPVIGTGIG |
| | (B12) | | GAIGGVSGGLTGAATFC |
| 27 | mutant 24 (C1) | aa | GWWNSWGKCVAGTIGGAGTGGLGGAAAGAAVPVIGTGIG |
| | | | GAIGGVSGGLTGAATFC |
| 28 | mutant 25 (C2) | aa | GWWNSWGKCVAGTIGGAGTGGLGGAAAGSAAPVIGTGIG |
| | | | GAIGGVSGGLTGAATFC |
| 29 | mutant 26 (C3) | aa | GWWNSWGKCVAGTIGGAGTGGLGGAAAGSAVAVIGTGIG |
| | | | GAIGGVSGGLTGAATFC |
| 30 | mutant 27 (C4) | aa | GWWNSWGKCVAGTIGGAGTGGLGGAAAGSAVPAIGTGIG |
| | | | GAIGGVSGGLTGAATFC |
| 31 | mutant 28 (C5) | aa | GWWNSWGKCVAGTIGGAGTGGLGGAAAGSAVPVAGTGI |
| | | | GGAIGGVSGGLTGAATFC |
| 32 | mutant 29 (C6) | aa | GWWNSWGKCVAGTIGGAGTGGLGGAAAGSAVPVIATGIG |
| | | | GAIGGVSGGLTGAATFC |
| 33 | mutant 30 (C7) | aa | GWWNSWGKCVAGTIGGAGTGGLGGAAAGSAVPVIGAGIG |
| | | | GAIGGVSGGLTGAATFC |
| 34 | mutant 31 (C8) | aa | GWWNSWGKCVAGTIGGAGTGGLGGAAAGSAVPVIGTAIG |
| | | | GAIGGVSGGLTGAATFC |
| 35 | mutant 32 (C9) | aa | GWWNSWGKCVAGTIGGAGTGGLGGAAAGSAVPVIGTGA |
| | | | GGAIGGVSGGLTGAATFC |
| 36 | mutant 33 | aa | GWWNSWGKCVAGTIGGAGTGGLGGAAAGSAVPVIGTGIA |
| | (C10) | | GAIGGVSGGLTGAATFC |
| 37 | mutant 34 | aa | GWWNSWGKCVAGTIGGAGTGGLGGAAAGSAVPVIGTGIG |
| | (C11) | | AAIGGVSGGLTGAATFC |
| 38 | mutant 35 | aa | GWWNSWGKCVAGTIGGAGTGGLGGAAAGSAVPVIGTGIG |
| | (C12) | | GAAGGVSGGLTGAATFC |
| 39 | mutant 36 (D1) | aa | GWWNSWGKCVAGTIGGAGTGGLGGAAAGSAVPVIGTGIG |
| | | | GAIAGVSGGLTGAATFC |
| 40 | mutant 37 (D2) | aa | GWWNSWGKCVAGTIGGAGTGGLGGAAAGSAVPVIGTGIG |
| | | | GAIGAVSGGLTGAATFC |
| 41 | mutant 38 (D3) | aa | GWWNSWGKCVAGTIGGAGTGGLGGAAAGSAVPVIGTGIG |
| | | | GAIGGASGGLTGAATFC |
| 42 | mutant 39 (D4) | aa | GWWNSWGKCVAGTIGGAGTGGLGGAAAGSAVPVIGTGIG |
| | | | GAIGGVAGGLTGAATFC |
| 43 | mutant 40 (D5) | aa | GWWNSWGKCVAGTIGGAGTGGLGGAAAGSAVPVIGTGIG |
| | | | GAIGGVSAGLTGAATFC |
| 44 | mutant 41 (D6) | aa | GWWNSWGKCVAGTIGGAGTGGLGGAAAGSAVPVIGTGIG |
| | | | GAIGGVSGALTGAATFC |
| 45 | mutant 42 (D7) | aa | GWWNSWGKCVAGTIGGAGTGGLGGAAAGSAVPVIGTGIG |
| | | | GAIGGVSGGATGAATFC |
| 46 | mutant 43 (D8) | aa | GWWNSWGKCVAGTIGGAGTGGLGGAAAGSAVPVIGTGIG |
| | | | GAIGGVSGGLAGAATFC |
| 47 | mutant 44 (D9) | aa | GWWNSWGKCVAGTIGGAGTGGLGGAAAGSAVPVIGTGIG |
| | | | GAIGGVSGGLTAAATFC |
| 48 | mutant 45 | aa | GWWNSWGKCVAGTIGGAGTGGLGGAAAGSAVPVIGTGIG |
| | (D10) | | GAIGGVSGGLTGAAAFC |
| 49 | mutant 46 | aa | GWWNSWGKCVAGTIGGAGTGGLGGAAAGSAVPVIGTGIG |
| | (D11) | | GAIGGVSGGLTGAATAC |
| 50 | mutant 47 | aa | GWWNSWGKCVAGTIGGAGTGGLGGAAAGSAVPVIGTGIG |
| | (D12) | | GAIGGVSGGLTGAATFA |
| 51 | mutant 48 (E2) | aa | GWWNSWGKCVAGTIGGAGTGGLGGAAAGSAVPVIGTGIG |
| | | | GAIGGVSGGLTGAATFCW |
| 52 | class II | aa | GGWWNSWGKCAAGTLGGTGTGVLGGAAVGSSVPVIGTG |
| | bacteriocin | | IGAAVGGVSGALTGAASAC |
| 53 | class II | aa | GGWWSSWGKCAAGTLGGTGTGVLGGAAVGSSVPVIGTGI |
| | bacteriocin | | GAAVGGVSGALTGAASAC |
| 54 | class II | aa | GGGWWSSWGKCAAGTLGGTGTGALGGAAVGSSVPVIGT |
| | bacteriocin | | GIGAAVGGVSGALTGAAASC |
| 55 | cerein 7B | DNA | GCATGCAAGGAGATGGCGCCCAACAGTCCCCCGGCCA |
| | | | CGGGGCCTGCCACCATACCCACGCCGAAACAAGCGCTC |
| | | | ATGAGCCCGAAGTGGCGAGCCCGATCTTCCCCATCGGT |
| | | | GATGTCGGCGATATAGGCGCCAGCAACCGCACCTGTGG |
| | | | CGCCGGTGATGCCGGCCACGATGCGTCCGGCGTAGAG |
| | | | GATCGGATCTCGATCCCGCGAAATTAATACGACTCACTA |
| | | | TAGGGGAATTGTGAGCGGATAACAATTCCCCTCTAGAAA |
| | | | TAATTTTGTTTAACTTTAAGAAGGAGATATACATATGGGC |
| | | | TGGTGGAACAGCTGGGGTAAATGTGTTGCTGGAACTAT |
| | | | CGGTGGAGCTGGAACTGGTGGTTTAGGTGGAGCTGCTG |
| | | | CAGGTTCAGCTGTTCCGGTTATTGGTACTGGTATTGGTG |
| | | | GCGCTATTGGTGGAGTTAGCGGTGGCCTTACAGGTGCA |
| | | | GCTACTTTTTGCTAGGATCCATCCGGCTGCTAACAAAGC |
| | | | CCGAAAGGAAGCTGAGTTGGCTGCTGCCACCGCTGAGC |
| | | | AATAACTAGCATAACCCCTTGGGGCCTCTAAACGGGTCT |
| | | | TGAGGGGTTTTTTGCTGAAAGGAGGAACTATATCCGGAT |
| | | | TCAGGAGAGCGTTCACCGACAAACAACAGATAAAACGA |
| | | | AAGGCCCAGTCTTTCGACTGAGCCTTTCGTTTTATTTGA |
| | | | TGCCTGGCAGTTCCCTACTCTCGCATCACGTG |
| 56 | motif | aa | GWGDVL |

### REFERENCES

Shorr A F. Epidemiology and economic impact of meticillin-resistant Staphylococcus aureus: review and analysis of the literature. Pharmacoeconomics 445; 25:751-68, 2007.
Chan L. C., et al. Ceftobiprole- and ceftaroline-resistant methicillin-resistant Staphylococcus aureus. Antimicrob Agents Chemother 59:2960-3, 2015.
Mc Guinness W. A., et al. Vancomycin Resistance in Staphylococcus aureus. Yale J Biol Med 90(2):269-281, 2017.
Ovchinnikov, K. V., et al. Successful development of bacteriocins into therapeutic formulation for treatment of MRSA-skin infection in a murine model. AAC 17; 64(12):e00829-20, (2020). Byrd, A., et al. The human skin microbiome. Nat Rev Microbiol 16:143-155 (2018).
Nakatsuji, T. et al. Antimicrobials from human skin commensal bacteria protect against Staphylococcus aureus and are deficient in atopic dermatitis. Sci Transl Med 9:eaah4680 (2017).
Gabant P., Borrero J. PARAGEN 1.0: A standardized synthetic gene library for fast cell-free bacteriocin synthesis. Front. Bioeng. Biotechnol. 7:213 (2019).
Oscáriz J. C., et al. Purification and sequence of cerein 7B, a novel bacteriocin produced by Bacillus cereus Bc7. FEMS Microbiol. Lett. 254: 108-115 (2006).

1. A bacteriocin peptide or peptidomimetic, wherein said peptide or peptidomimetic comprises, essentially consists of, or consists of a peptide or peptidomimetic represented by the amino acid sequence of SEQ ID NO: 2, or by an amino acid sequence having at least 60%, 70%, 80%, 85%, 90%, 95%, or 99% identity or similarity with SED ID NO: 2, for use in the treatment, prevention and/or delaying of an infection on a subject, preferably a human subject, wherein the infection is:
a. a skin infection and/or
b. caused by Staphylococcus aureus.

2. A bacteriocin peptide or peptidomimetic for use according to claim 1, wherein the skin infection is caused by a bacterium, preferably a Gram-positive bacterium.

3. A bacteriocin peptide or peptidomimetic for use according to claim 2, wherein the bacterium is Staphylococcus aureus.

4. A bacteriocin peptide or peptidomimetic for use according to claim 1 or 3, wherein the Staphylococcus aureus is methicillin-resistant.

5. A bacteriocin peptide or peptidomimetic for use according to any one of claims 1-4, wherein said use does not inhibit at least one of the microbial cells normally present on the subject's skin, preferably Staphylococcus epidermidis.

6. A composition comprising a bacteriocin peptide or peptidomimetic as defined in any one of claims 1-5.

7. A composition according to claim 6, wherein said composition is a pharmaceutical composition optionally further comprising one or more antimicrobial compounds and/or pharmaceutically acceptable ingredients.

8. A pharmaceutical composition according to claim 7 for use according to any one of claims 1-5.

9. A bacteriocin peptide or peptidomimetic for use according to any one of claims 1-5, or a pharmaceutical composition for use according to claim 8, wherein said peptide, peptidomimetic, or composition is administered topically or transdermally, preferably on a wound, lesion, or abscess.

10. A composition according to claim 6, wherein said composition is a cosmetic composition optionally further comprising one or more antimicrobial compounds and/or cosmetically acceptable ingredients.

11. A cosmetic method of providing an improvement of skin hygiene comprising applying to the skin a bacteriocin peptide or peptidomimetic as defined in any one of claims 1-5, or a cosmetic composition as defined in claim 10.

12. A composition according to claim 6, wherein said composition comprises a bacteriocin peptide or peptidomimetic as defined in claim 1 or 4, wherein said composition is suitable for disinfecting a surface contaminated with Staphylococcus aureus, optionally further comprising one or more antimicrobial compounds and/or a solvent.

13. An ex-vivo method of disinfecting a surface comprising contacting said surface with a bacteriocin peptide or peptidomimetic as defined in claim 1 or 4, or with a composition as defined in claim 12.

14. A bacteriocin peptide or peptidomimetic, wherein said peptide or peptidomimetic comprises, essentially consists of, or consists of a peptide or peptidomimetic represented by the amino acid sequence of any one of SEQ ID NOs: 7, 8, 15, 18, 19, 22, 24, 34, 37, 40, 42, 44, 47, 48, or 51.
